(19) United States
(12) Patent Application Publication
(10) Pub. No.: US 2025/0312010 A1
(43) Pub. Date: Oct. 9, 2025

(54) ULTRASOUND IMAGING METHOD AND ULTRASOUND IMAGING SYSTEM

(71) Applicant: SHENZHEN MINDRAY BIO-MEDICAL ELECTRONICS CO., LTD., Shenzhen (CN)

(72) Inventors: Longlong HOU, Shenzhen (CN); Chongchong GUO, Shenzhen (CN); Feng WANG, Shenzhen (CN); Lei LI, Shenzhen (CN)

(73) Assignee: SHENZHEN MINDRAY BIO-MEDICAL ELECTRONICS CO., LTD., Shenzhen (CN)

(21) Appl. No.: 19/171,708

(22) Filed: Apr. 7, 2025

(30) Foreign Application Priority Data
Apr. 7, 2024 (CN) 202410411931.4

(51) Int. Cl. A61B 8/00; G16H 30/40

(52) U.S. Cl. CPC A61B 8/463; A61B 8/469; A61B 8/5207; A61B 8/5246; A61B 8/54; G16H 30/40

(57) ABSTRACT

An ultrasonic imaging method includes: controlling an ultrasonic probe to transmit first and second ultrasonic waves to first and second regions of the same tissue imaging plane, respectively and to receive echo signals of the first and second ultrasonic waves; generating a first ultrasonic image and a second ultrasonic image based on the echo signals of the first and second ultrasonic waves, respectively; and displaying the two images on first and second display regions of a display interface, respectively; wherein the first and second ultrasonic images are of the same image mode, and the image display characteristic of the second ultrasonic image is higher than the corresponding one of the first ultrasonic image. The contrast between the two real-time ultrasonic images is thereby enhanced as disclosed herein.

### CROSS-REFERENCE TO RELATED APPLICATION

The present application claims priority to Chinese Patent Application No. 202410411931.4 filed on Apr. 7, 2024, the disclosure of which is hereby incorporated by reference in its entirety.

### BACKGROUND

Ultrasonic imaging technology enables the imaging of numerous organs or tissues within a human body, assisting doctors in making diagnosis. This technology utilizes ultrasonic waves to scan a human body's tissues and organs, receiving and processing reflected signals to obtain images of corresponding regions. Specifically, it involves transmitting ultrasonic waves to a target tissue, receiving echo data reflected by the tissue, and generating an ultrasonic image of the target tissue based on the received echo data. Due to its advantages of non-invasive, low cost and high real-time performance, ultrasonic imaging has gradually emerged as the most widely used and frequently utilized imaging modality in medical imaging examinations.

In order to acquire more information, a dual real-time ultrasonic imaging technique has emerged, which displays two real-time ultrasonic images simultaneously on the left and right sides of a display, allowing users to observe additional details and conveniently compare the two real-time ultrasonic images. Additionally, image processing/optimization is typically performed on each real-time ultrasonic image to enhance the contrast between the two.

Currently, there is still room for improvement in dual real-time ultrasonic imaging technology, such as the need to further enhance the contrast.

### SUMMARY

In view of the aforementioned issues, the present disclosure provides ultrasonic imaging methods and ultrasonic imaging systems, as described in detail below.

The present disclosure relates to ultrasonic imaging, in particular to ultrasonic imaging methods and ultrasonic imaging systems.

In accordance with a first aspect, an ultrasonic imaging method provided in some embodiments may include:
controlling an ultrasonic probe to transmit first ultrasonic waves to a first region of a tissue and receive echo signals of the first ultrasonic waves, and generating a first ultrasonic image based on the echo signals of the first ultrasonic waves;
displaying the first ultrasonic image on a first display region of a display interface;
obtaining a second region from the first region based on the first ultrasonic image;
controlling the ultrasonic probe to alternately transmit the first ultrasonic waves and the second ultrasonic waves to the first region and the second region for a plurality of times and receive the echo signals of the first ultrasonic waves and echo signals of the second ultrasonic waves, updating the first ultrasonic image based on the echo signals of the first ultrasonic waves, and generating a second ultrasonic image based on the echo signals of the second ultrasonic waves; and
displaying the updated first ultrasonic image on the first display region of the display interface, and displaying the second ultrasonic image on the second display region of the display interface;
wherein the first display region and the second display region are two different display regions;
the first ultrasonic image and the second ultrasonic image are of the same image mode; and
the image display characteristic of the second ultrasonic image is higher than the corresponding image display characteristic of the second region in the first ultrasonic image, with the image display characteristic comprising at least one of an image penetration, an image contrast, an image resolution, an image clarity, and a richness of image details.

In some embodiments,
the ultrasonic probe is controlled to transmit first ultrasonic waves according to a first transmission parameter and the ultrasonic probe is controlled to transmit the second ultrasonic waves according to a second transmission parameter, with the first transmission parameter being the same as the second transmission parameter;
the generation and/or update of the first ultrasonic image based on the echo signals of the first ultrasonic waves comprises: performing a first processing on the echo signals of the first ultrasonic waves; and the generation of the second ultrasonic image based on the echo signals of the second ultrasonic waves comprises: performing a second processing on the echo signals of the second ultrasonic waves; where
the first processing comprises a first signal processing and/or a first image processing, the second processing comprises a second signal processing and/or a second image processing; and
the first signal processing is different from the second signal processing,
and/or the first image processing is different from the second image processing.

In some embodiments,
the first signal processing comprises a first beam forming procedure, and the second signal processing comprises a second beam forming procedure, with the first beam forming procedure being different from the second beam forming procedure; and/or
the first signal processing comprises a first signal demodulation procedure, and the second signal processing comprises a second signal demodulation procedure, with the first signal demodulation procedure being different from the second signal demodulation procedure.

In some embodiments, the ultrasonic probe is controlled to transmit the first ultrasonic waves according to a first transmission parameter, and the ultrasonic probe is controlled to transmit the second ultrasonic waves according to a second transmission parameter, with the first transmission parameter being different from the second transmission parameter.

In some embodiments, the first transmission parameter and the second transmission parameter comprise at least one of a pulse amplitude, a transmit voltage, a transmit frequency, a number of transmissions, a transmit interval, a transmit angle, a transmit waveform, a transmit aperture, a line density, a pixel density, and a focal position.

In some embodiments, the first transmission parameter and the second transmission parameter are configured such that the scan density of the second region by the second ultrasonic waves is greater than the scan density of the first region by the first ultrasonic waves, with the scan density comprising a scanning line density and/or a scanning pixel density.

In some embodiments, the image mode is one of a two-dimensional grayscale image, a Doppler image, a vector flow image, a superb microvascular image, a contrast-enhanced ultrasonic image, a photoacoustic image, and an elasticity image.

In some embodiments, the obtaining of a second region from the first region based on the first ultrasonic image comprises:
in response to a user operation, obtaining a sampling region from the first ultrasonic image displayed on the first display region; and
calculating the second region based on the sampling region.

In some embodiments,
in response to a/the user operation, the image display characteristic of the second ultrasonic image is selected from a plurality of image display characteristics; and
according to the selected image display characteristic, the second processing associated with said image display characteristic is determined, with different image display characteristics being associated with different second processing; and/or, according to the selected image display characteristic, the second transmission parameter associated with said image display characteristic is determined, with different image display characteristics being associated with different second transmission parameters.

In accordance with a second aspect, an ultrasonic imaging method provided in some embodiments may include:
controlling an ultrasonic probe to transmit first and second ultrasonic waves to a first region and a second region of a same tissue imaging plane, respectively, and receive echo signals of the first and second ultrasonic waves;
generating a first ultrasonic image based on the echo signals of the first ultrasonic waves, and generating a second ultrasonic image based on the echo signals of the second ultrasonic waves; and
displaying the first ultrasonic image and the second ultrasonic image on a first display region and a second display region of a display interface, respectively; wherein
the first display region and the second display region are different,
the first ultrasonic image and the second ultrasonic image are of the same image mode, and
the image display characteristic of the second ultrasonic image is higher than the corresponding image display characteristic of the first ultrasonic image, with the image display characteristic comprising at least one of an image penetration, an image contrast, an image resolution, and an image clarity.

In some embodiments,
the ultrasonic probe is controlled to transmit the first ultrasonic waves according to a first transmission parameter, and the ultrasonic probe is controlled to transmit the second ultrasonic waves according to the second transmission parameter, with the first transmission parameter being the same as the second transmission parameter;
the generation of the first ultrasonic image based on the echo signals of the first ultrasonic waves comprises: performing a first processing on the echo signals of the first ultrasonic waves; and the generation of the second ultrasonic image based on the echo signals of the second ultrasonic waves comprises: performing a second processing on the echo signals of the second ultrasonic waves; where
the first processing comprises a first signal processing and/or a first image processing, and the second processing comprises a second signal processing and/or a second image processing;
the first signal processing is different from the second signal processing;
and/or the first image processing is different from the second image processing.

In some embodiments,
the first signal processing comprises a first beam forming procedure, and the second signal processing comprises a second beam forming procedure, with the first beam forming procedure being different from the second beam forming procedure;
and/or the first signal processing comprises a first signal demodulation procedure, and the second signal processing comprises a second signal demodulation procedure, with the first signal demodulation procedure being different from the second signal demodulation procedure.

In some embodiments,
the ultrasonic probe is controlled to transmit the first ultrasonic waves according to a first transmission parameter, and the ultrasonic probe is controlled to transmit the second ultrasonic waves according to a second transmission parameter, with the first transmission parameter being different from the second transmission parameter.

In some embodiments, the first transmission parameter and the second transmission parameter comprise at least one of a pulse amplitude, a transmit voltage, a transmit frequency, a number of transmissions, a transmit interval, a transmit angle, a transmit waveform, a transmit aperture, a line density, a pixel density, and a focal position.

In some embodiments, the second region is a sub-region of the first region.

In some embodiments, the first transmission parameter and the second transmission parameter are configured such that the scan density of the second region by the second ultrasonic waves is greater than the scan density of the first region by the first ultrasonic waves, with the scan density comprising a scanning line density and/or a scanning pixel density.

In some embodiments, the frame rate of the second ultrasonic image is greater than that of the first ultrasonic image.

In some embodiments, the first region is the same as the second region.

In some embodiments, the transmit frequency of the first transmission parameter is lower than the transmit frequency of the second transmission parameter.

In some embodiments, the first region and the second region are different.

In some embodiments, the first transmission parameter and the second transmission parameter enable the ultrasonic probe to perform a vector scan and a linear scan, respectively.

In some embodiments,
in response to a user operation, the image display characteristic of the first ultrasonic image is selected from a plurality of image display characteristics; and
according to the selected image display characteristic, the first processing associated with said image display characteristic is determined, with different image display characteristics being associated with different first processing; and/or, according to the selected image display characteristic, the first transmission parameter associated with said image display characteristic is determined, with different image display characteristics being associated with different first transmission parameters.

In some embodiments,
in response to a user operation, the image display characteristic of the second ultrasonic image is selected from a plurality of image display characteristics; and
according to the selected image display characteristic, the second processing associated with said image display characteristic is determined, with different image display characteristics being associated with different second processing; and/or, according to the selected image display characteristic, the second transmission parameter associated with said image display characteristic is determined, with different image display characteristics being associated with different second transmission parameters.

In some embodiments, the first ultrasonic image and the second ultrasonic image are capable of being subjected to independent image manipulations respectively, said image manipulations comprising one or more of image rotation, image tilt, image inversion, image magnification, and image parameter measurement.

In some embodiments, the image mode is one of a two-dimensional grayscale image, a Doppler image, a vector flow image, a superb microvascular image, a contrast-enhanced ultrasonic image, a photoacoustic image, and an elasticity image.

In some embodiments, the ultrasonic imaging method may further include:
controlling the ultrasonic probe to transmit third ultrasonic waves to the second region and receive echo signals of the third ultrasonic waves, generating a third ultrasonic image based on the echo signals of the third ultrasonic waves, with the third ultrasonic image and the second ultrasonic image being of different image modes; and
superimposing and displaying the third ultrasonic image on the second ultrasonic image, or displaying the third ultrasonic image on the third display region of the display interface, with the third display region being different from the first display region and the second display region.

In accordance with a third aspect, an ultrasonic imaging method provided in some embodiments may include:
controlling an ultrasonic probe to transmit first ultrasonic waves and second ultrasonic waves to a first region and a second region of the same tissue imaging plane, respectively, and receive echo signals of the first ultrasonic waves and echo signals of the second ultrasonic waves;
generating a first ultrasonic image based on the echo signals of the first ultrasonic waves, and generating a second ultrasonic image based on the echo signals of the second ultrasonic waves;
displaying the first ultrasonic image and the second ultrasonic image on a first display region and a second display region of a display interface, respectively; wherein
the first display region and the second display region are different,
the first ultrasonic image and the second ultrasonic image are of the same image mode, and
the image display characteristic of the second ultrasonic image is higher than the corresponding image display characteristic of the first ultrasonic image.

In accordance with a fourth aspect, an ultrasonic imaging method provided in some embodiments may include:
controlling an ultrasonic probe to transmit first ultrasonic waves to a tissue imaging plane and receive echo signals of the first ultrasonic waves;
performing a first signal processing on the echo signals of the first ultrasonic waves to generate a first ultrasonic image of a first region on the tissue imaging plane, and performing a second signal processing on the echo signals of the first ultrasonic waves to generate a second ultrasonic image of a second region on the tissue imaging plane; and
displaying the first ultrasonic image and the second ultrasonic image on a first display region and a second display region of a display interface, respectively; wherein
the first display region and the second display region are different,
the first ultrasonic image and the second ultrasonic image are of the same image mode, and
the image display characteristic of the second ultrasonic image is higher than the corresponding image display characteristic of the first ultrasonic image, with the image display characteristic comprising at least one of an image penetration, an image contrast, an image resolution, and an image clarity.

In accordance with a fifth aspect, an ultrasonic imaging system provided in some embodiments may include:
an ultrasonic probe, configured to transmit ultrasonic waves and receive echo signals of the ultrasonic waves;
a transmit and receive control circuit, configured to control the ultrasonic probe to perform transmission of the ultrasonic waves and reception of the echo signals of the ultrasonic waves; and
a processor, configured to perform the method described in any embodiment disclosed herein.

Based on the ultrasonic imaging methods and the ultrasonic imaging systems mentioned in the above embodiments, by configuring different transmission parameters, the echo signals of the first ultrasonic waves and the echo signals of the second ultrasonic waves are different, such that the echo signals of the second ultrasonic waves possess features that facilitate the presentation of the same image display characteristic in ultrasonic images, as compared to the echo signals of the first ultrasonic waves.

Based on the ultrasonic imaging methods and the ultrasonic imaging systems described in the above embodiments, by configuring different transmission parameters, the echo signals of the first ultrasonic waves and the echo signals of the second ultrasonic waves are different, which significantly increases the room for processing the two real-time ultrasonic images in subsequent signal and image processing, enabling greater differences in the same image display characteristic between the two real-time ultrasonic images.

Based on the ultrasonic imaging methods and the ultrasonic imaging systems described in the above embodiments, different signal processing is applied to the the echo signals of the first ultrasonic waves and the echo signals of the second ultrasonic waves respectively, such that the second ultrasonic image data obtained by processing the echo signals of the second ultrasonic waves with the second signal processing possesses features that facilitate the presentation of the same image display characteristic in ultrasonic images, as compared to the first ultrasonic image data obtained by processing the echo signals of the first ultrasonic waves with the first signal processing.

Based on the ultrasonic imaging methods and the ultrasonic imaging systems described in the above embodiments, by applying different signal processing to the echo signals of the first and second ultrasonic waves respectively, the flexibility in subsequent imaging processing is also effectively enhanced, allowing for greater differences in the same image display characteristics between the two real-time ultrasonic images.

### BRIEF DESCRIPTION OF THE DRAWINGS

FIG. 1 is a schematic diagram illustrating dual real-time ultrasonic imaging of two different cross-sections of a heart;

FIG. 2A is a schematic diagram illustrating dual real-time ultrasonic imaging of a single cross-section of the heart;

FIG. 2B is another schematic diagram illustrating dual real-time ultrasonic imaging of a single cross-section of the heart;

FIG. 3 is a schematic diagram illustrating the structure of an ultrasonic imaging system in some embodiments;

FIG. 4 is a schematic diagram illustrating the structure of an ultrasonic probe in some embodiments;

FIG. 5 is a schematic diagram illustrating the structure of an array of transducer elements in some embodiments;

FIG. 6 is a schematic diagram illustrating the structure of an array of transducer elements in some embodiments;

FIG. 7 is a schematic diagram illustrating the structure of an array of transducer elements in some embodiments;

FIG. 8 is a schematic diagram illustrating the structure of an array of transducer elements in some embodiments;

FIG. 9 is a schematic diagram illustrating the structure of an array of transducer elements in some embodiments;

FIG. 10 is a schematic diagram illustrating the structure of an array of transducer elements in some embodiments;

FIG. 11 is a schematic diagram illustrating the structure of an array of transducer elements in some embodiments;

FIG. 12 is a schematic diagram illustrating the structure of an array of transducer elements in some embodiments;

FIG. 13A is a schematic diagram illustrating an example of data rearrangement;

FIG. 13B is a schematic diagram illustrating another example of data rearrangement;

FIG. 13C is a schematic diagram illustrating yet another example of data rearrangement;

FIG. 14 is a schematic flowchart of an ultrasonic imaging method in some embodiments;

FIG. 15 is a schematic diagram illustrating a display interface in some embodiments;

FIG. 16 is a schematic partial flowchart of an ultrasonic imaging method in some embodiments;

FIG. 17 is a schematic partial flowchart of an ultrasonic imaging method in some embodiments;

FIG. 18 is a schematic flowchart of an ultrasonic imaging method in some embodiments;

FIG. 19 is a schematic flowchart of an ultrasonic imaging method in some embodiments;

FIG. 20 is a schematic diagram illustrating a first region in some embodiment;

FIG. 21 is a schematic flowchart of an ultrasonic imaging method in some embodiments; and

FIG. 22 is a schematic partial flowchart of an ultrasonic imaging method in some embodiments.

### DETAILED DESCRIPTION

The present disclosure will be further described in detail below through specific embodiments with reference to the accompanying drawings. Common or similar elements are referenced with like or identical reference numerals in different embodiments. Many details described in the following embodiments are for better understanding the present disclosure. However, those skilled in the art can realize with minimal effort that some of these features can be omitted in different cases or be replaced by other elements, materials and methods. For clarity some operations related to the present disclosure are not shown or illustrated herein so as to prevent the core from being overwhelmed by excessive descriptions. For those skilled in the art, such operations are not necessary to be explained in detail, and they can fully understand the related operations according to the description in the specification and the general technical knowledge in the art.

In addition, the features, operations or characteristics described in the specification may be combined in any suitable manner to form various embodiments. At the same time, the steps or actions in the described method can also be sequentially changed or adjusted in a manner that can be apparent to those skilled in the art. Therefore, the various sequences in the specification and the drawings are only for the purpose of describing a particular embodiment, and are not intended to be an order of necessity, unless otherwise stated one of the sequences must be followed.

The serial numbers of components herein, such as “first”, “second”, etc., are only used to distinguish the described objects and do not have any order or technical meaning. The terms “connected”, “coupled” and the like here include direct and indirect connections (coupling) unless otherwise specified.

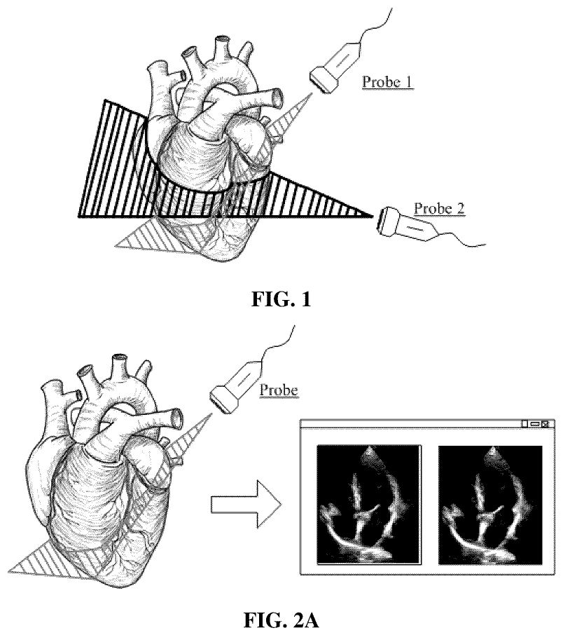

A common application scenario of dual real-time ultrasonic imaging technology at present is to perform real-time ultrasonic imaging on multiple different cross-sections of an organ or tissue and display real-time ultrasonic images of the same image mode (e.g., B-mode images) for these different cross-sections. For example, the heart has various sections such as the left ventricular long-axis section, the right ventricular inflow tract long-axis section, the right ventricular outflow tract long-axis section, another left ventricular long-axis section (if distinguished by different imaging planes or views), the left ventricular outflow tract horizontal short-axis section, the aortic root short-axis section and the pulmonary artery bifurcation horizontal short-axis section. When performing real-time ultrasonic imaging on multiple different cross-sections of an organ or tissue, it is typically necessary to use multiple probes/transducers, with each probe/transducer corresponding to one cross-section. This allows multiple probes/transducers to simultaneously perform real-time imaging of multiple different cross-sections of the organ or tissue, and concurrently display real-time ultrasonic images (e.g., B-mode images) of these different cross-sections, facilitating users to compare different cross-sections of the organ or tissue. For example, FIG. 1 illustrates an example where two ultrasonic probes, namely Probe 1 and Probe 2 in the figure, perform real-time ultrasonic imaging on two different cross-sections of the heart. The cross-section corresponding to Probe 1 is indicated by the area outlined and filled in with black line in the figure; while the cross-section corresponding to Probe 2 is indicated by the area outlined and filled in with gray line in the figure.

Another common application scenario of dual real-time ultrasonic imaging technology is to perform real-time ultrasonic imaging on a single cross-section of an organ or tissue to obtain a plurality of (e.g., two) real-time ultrasonic images of the same image mode (e.g., B-mode images) for that same cross-section and display them. It can be seen that, since imaging is performed on a single cross-section of an organ or tissue, there is no need to use multiple probes/transducers. Additionally, when performing ultrasonic imaging on a cross-section of a patient's organ or tissue, only one probe can be placed in the appropriate area on the patient's body surface where the probe is to be contacted/positioned, making it impossible to use multiple probes. Therefore, users employ a single probe/transducer to perform dual real-time ultrasonic imaging on a cross-section of an organ or tissue. A typical process involves the following steps: an ultrasonic imaging system uses a single probe/transducer to emit ultrasonic waves towards a cross-section of an organ or tissue and receive corresponding echo signals, performs signal processing on the echo signals in the signal domain (such as analog-to-digital conversion, signal demodulation, amplification, filtering, and beamforming) to obtain ultrasonic image data, and then preforms image processing (including scan conversion, dynamic range adjustment, frame correlation, smoothing, and image enhancement) on the ultrasonic image data in the image domain to obtain a plurality of ultrasonic images for display on a monitor (e.g., displaying two ultrasonic images on the monitor). The plurality of ultrasonic images are obtained by processing the same ultrasonic image data through different image processing procedures (including different image processing procedures and/or different image processing parameters).

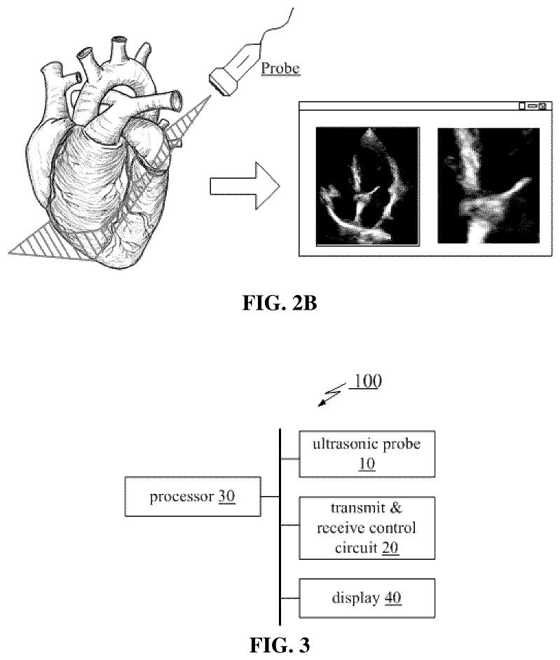

For example, FIG. 2A illustrates a case where two real-time ultrasonic images of the same type (e.g., B-mode images) depicting the same region of the same cross-section are displayed on the left and right sides of a monitor, respectively. Both images are obtained from the same ultrasonic image data but processed using different image processing procedures. For instance, the real-time ultrasonic image on the left is not smoothed, whereas the one on the right is smoothed. Another example could be that the parameters related to dynamic range applied to the real-time ultrasonic image on the left are different from those applied to the image on the right. By applying different image processing procedures to the same ultrasonic image data, the plurality of real-time ultrasonic images can exhibit various display effects, facilitating users in comparing and observing.

For another example, FIG. 2B illustrates a case where a real-time ultrasonic image of a region on a cross-section is displayed on the left side of a monitor. Since a specific small sub-area (depicted as the area near the center of the left image in the schematic of FIG. 2B) within this region is of particular interest to users and requires further observation, the real-time ultrasonic image of this small sub-area is displayed on the right side of the monitor. Additionally, to facilitate better observation and comparison, it may be necessary to apply different image processing procedures to the ultrasonic image corresponding to this small sub-area. In this case, both the left and right real-time ultrasonic images are from the same ultrasonic image data source, with the exception that the real-time ultrasonic image on the right only requires a portion of the ultrasonic image data.

It shall be noted that the ultrasonic images shown in FIG. 2A and FIG. 2B are for illustration and explanation purposes only, and are not intended to limit the actual ultrasonic images corresponding to the cardiac section depicted in the figure.

It can be seen that real-time ultrasonic imaging and display of multiple different cross-sections of an organ or tissue facilitate users in comparing these different cross-sections of the organ or tissue, thus necessitating the use of multiple probes/transducers. On the other hand, dual real-time imaging of a single cross-section of an organ or tissue employs different image processing procedures to facilitate users in observing and comparing regions of the same cross-section of the organ or tissue. This is because different diagnostic information that users want to observe and understand from ultrasonic images may need to be highlighted under different imaging effects.

However, the inventor has found that the contrast of dual real-time imaging for a cross-section of an organ or tissue at present limited; also, the potential of the mainstream improvement approach, which relies on researching and developing image processing algorithms, is constrained. The inventor believes that this current improvement approach essentially still relies on applying different image processing to dual real-time ultrasonic images, with a limited range of adjustable parameters, thus limiting its development potential. The inventor proposes that enhancing the contrast of dual real-time imaging for the same cross-section of an organ or tissue should not be limited to applying different imaging processing procedures to the dual real-time ultrasonic images. Instead, this can be achieved by implementing diverse signal processing methods on the received echo signals at the front-end, or even by designing and arranging distinct ultrasonic scanning sequences for the two real-time ultrasonic images of the same cross-section using a single ultrasonic probe, resulting in different front-end transmission parameters. This greatly increases the flexibility in processing the two real-time ultrasonic images, thereby effectively enhancing the contrast of dual real- time imaging.

Next, an ultrasonic imaging system will be described.

Please refer to FIG. 3. The ultrasonic imaging system 100 in some embodiments may include an ultrasonic probe 10, a transmit and receive control circuit 20 and a processor 30. In some embodiments, the ultrasonic imaging system 100 may also include a display 40. The following is a description of these components.

In some embodiments, the ultrasonic probe 10 is configured to transmit ultrasonic waves and receive echo signals of the ultrasonic waves. In some specific embodiments, the ultrasonic probe 10 comprises a plurality of transducer elements for mutual conversion between electrical pulse signals and ultrasonic waves, thereby enabling the transmission of ultrasonic waves to a target region and the reception of ultrasonic echoes from tissues so as to obtain the echo signals of the ultrasonic waves. In some embodiments, the plurality of transducer elements included in the ultrasonic probe 10 can be arranged in a single row to form a linear array. In some embodiments, the plurality of transducer elements included in the ultrasonic probe 10 are arranged in a two-dimensional matrix to form a planar array. The transducer elements, which for example employ piezoelectric crystals, convert electrical signals into ultrasonic signals according to a transmission sequence from the transmit and receive control circuit 20. Depending on the application, the transmitted ultrasonic waves (ultrasonic signals) may include one or more scanning pulses, one or more reference pulses, one or more push pulses, and/or one or more Doppler pulses. According to the wave form, the ultrasonic signals include focused waves, plane waves, and divergent waves. The transducer elements are used to transmit ultrasonic waves based on excitation electrical signals, or to convert received ultrasonic waves into electrical signals; accordingly, each transducer element can be used to achieve mutual conversion between electrical pulse signals and ultrasonic waves, enabling the transmission of ultrasonic waves to a target region and the reception of echo signals of ultrasonic waves from tissues. During ultrasonic inspection, the transmit and receive control circuit 20 can control which transducer elements are used to transmit ultrasonic beams (referred to as transmitting transducer elements), which transducer elements are used to receive ultrasonic beams (referred to as receiving transducer elements), or control the transducer elements to be used in time slots for emitting ultrasonic waves or receiving echoes of the ultrasonic waves. The transducer elements involved in transmission of ultrasonic waves can be simultaneously excited by electrical signals to transmit ultrasonic waves concurrently; alternatively, they can be excited by a plurality of electrical signals with certain time intervals to continuously transmit ultrasonic waves with specific time intervals. If the minimum processing area for receiving and reflecting ultrasonic waves in the target region is referred to as a location point within the tissue, then after the ultrasonic waves reach each location point in the target region, they will produce different reflections due to the different acoustic impedances of the tissues at different location points. The reflected ultrasonic waves are picked up by the receiving transducer elements, and each receiving transducer element may receive echoes of ultrasonic waves (i.e., ultrasonic echoes) from multiple location points. The ultrasonic echoes from different location points received by each receiving transducer element may form different channel echo data. For a particular receiving transducer element, its distances to different location points in the target region vary, so the times at which the ultrasonic echoes reflected from various location points arrive at the transducer element also differ. The correspondence between the ultrasonic echoes and the location points can be identified based on the arrival times of the ultrasonic echoes at said particular transducer element.

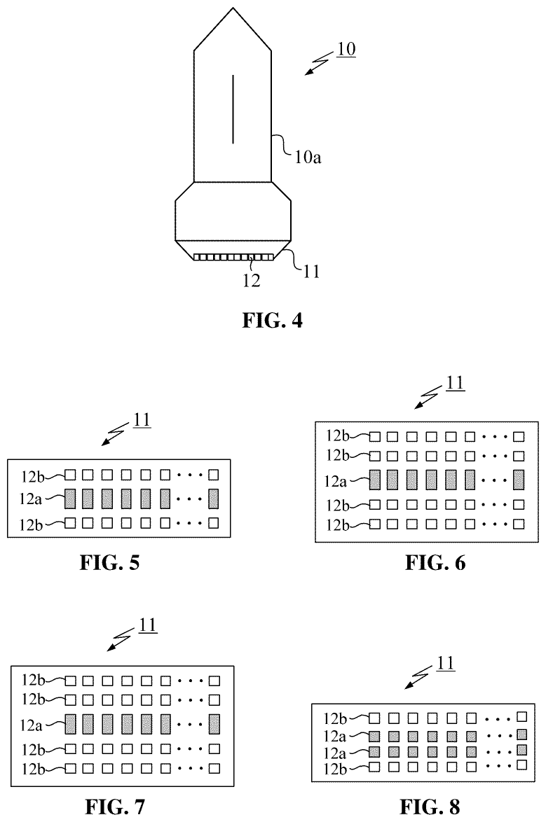

In some embodiments, the ultrasonic probe 10 comprises an array of transducer elements 11; and in some embodiments, the array of transducer elements 11 includes a plurality of transducer elements 12. FIG. 4 is an example, in which the ultrasonic probe 10 includes a probe body 10a and an array of transducer elements 11 disposed on the probe body 10a.

In some embodiments, the array of transducer elements 11 comprises at least two types of transducer elements 12 operating at different frequencies, for example, the array of transducer elements 11 includes several first-frequency transducer elements 12a and several second-frequency transducer elements 12b. Relevant examples are illustrated in FIGS. 5, 6, 7 and 8 below. It shall be understood that “several” as used herein refers to an indeterminate number, meaning one or more. In some embodiments, the transducer element 12 is configured to achieve mutual conversion between electrical signals and ultrasonic waves, thereby transmitting ultrasonic waves to the target region and receiving the echo signals reflected back by the tissue. The transducer elements can be implemented using, for example, piezoelectric ceramics, pressure crystals, or composite piezoelectric materials. The transducer elements are configured to emit ultrasonic waves based on excitation electrical signals, or to convert received ultrasonic waves into electrical signals. Accordingly, each transducer element can be configured to achieve the mutual conversion between electrical pulse signals and ultrasonic waves, thereby transmitting ultrasonic waves to the target region, and can also be configured to receive ultrasonic echo signals reflected back by tissues.

In some embodiments, the array of transducer elements 11 comprises transducer elements arranged in M rows and N columns, where M is an integer greater than or equal to 3, and N is an integer greater than or equal to 2. In some embodiments, for each column of transducer elements 12: the symmetrical transducer elements 12 within the column are connected in pairs, and a wire is led out from each pair to receive excitation signals from the same channel 01; and in the case where there is a single central transducer element 12 in the column, this central transducer element 12 is connected to a separate wire for connection to a channel and to receive excitation signals from that channel 01. In the transducer elements 12 arranged in M rows and N columns, there are a plurality of first-frequency transducer elements 12a and a plurality of second-frequency transducer elements 12b, and the transducer elements 12 connected to the same wire are of the same frequency.

In some embodiments, among the M rows and N columns of transducer elements 12, several rows of transducer elements 12 are first-frequency transducer elements 12a, and several rows of transducer elements 12 are second-frequency transducer elements 12b. That is, among the M rows and N columns of transducer elements 12, there is one or more rows of first-frequency transducer elements 12a and one or more rows of second-frequency transducer elements 12b. FIGS. 5, 6, 7 and 8 show four examples, where the gray-filled squares represent the first-frequency transducer elements 12a and the white-filled squares represent the second-frequency transducer elements 12b. In the example of FIG. 3, M=3, meaning that the array of transducer elements 11 has three rows of transducer elements, with the centrally located second row being the first-frequency transducer elements 12a, and the symmetrical first and third rows being the second-frequency transducer elements 12b. In the example of FIG. 4, M=5, meaning that the array of transducer elements 11 has five rows of transducer elements, with the centrally located third row being the first-frequency transducer elements 12a, the symmetrical first and fifth rows, along with the symmetrical second and fourth rows, being the second-frequency transducer elements 12b. In the example of FIG. 5, M=5, meaning that the array of transducer elements 11 has five rows of elements, with the centrally located third row, along with the symmetrical second and fourth rows, being the first-frequency transducer elements 12a, and the symmetrical first and fifth rows being the second-frequency transducer elements 12b. In the example of FIG. 6, M=4, meaning that the array of transducer elements 11 has four rows of transducer elements, with the symmetrical second and third rows being the first-frequency transducer elements 12a, and the symmetrical first and fourth rows being the second-frequency transducer elements 12b. The symmetry referred to here is in the column direction.

In some embodiments, M is an odd number; among the transducer elements arranged in M rows and N columns, the transducer elements 12 in the (M+1)/2nd row are the first-frequency transducer elements 12a, and the transducer elements 12 in the 1st row and the Mth row are the second-frequency transducer elements 12b. In some embodiments, the transducer elements 12 in the 1st to mth rows, as well as those in the (M+1−m)th to Mth rows, are the second-frequency transducer elements 12b, and the transducer elements 12 in the (m+1)th to (M−m)th rows are the first-frequency transducer elements 12a; where m is a positive integer and less than (M+1)/2.

In some embodiments, M is an odd number. In the case where M is odd, the transducer elements 12 in the (M+1)/2-th row constitute the central row. For each column of the transducer elements 12, the transducer element in the (M+1)/2-th row is the central transducer element of that column. Accordingly, for each column of the transducer element 12, the transducer element 12 located in the (M+1)/2-th row in that column is the central transducer element of that column, and it is individually connected to a wire for connecting to a channel 01 and receiving an excitation signal from that channel 01; and the transducer elements 12 in that column, which are symmetrically arranged around the central transducer element of that column (i.e., the transducer element in the (M+1)/2-th row), are connected in pairs and then connected to a wire to receive an excitation signal from the same channel 01. That is, the rows numbered from 1 to [(M+1)/2−1] in ascending order are symmetrical to the rows numbered from M to [(M+1)/2+1] in descending order.

In some embodiments, the transducer elements 12 in each pair of symmetrical rows are elements of the same frequency.

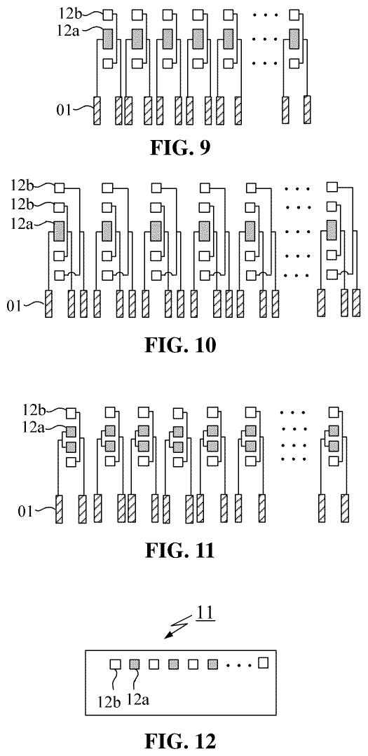

FIGS. 9 and 10 illustrate two examples. In the figures, the gray-filled squares represent the first-frequency transducer elements 12a, the white-filled squares represent the second-frequency transducer elements 12b, and the rectangles filled with diagonal lines represent the channel 01.

In the example of FIG. 9, M=3, meaning that the array of transducer elements 11 comprises three rows of transducer elements, with the transducer elements 12 in the second row being the transducer elements of the central row. For each column of transducer elements 12, the transducer elements 12 in the second row of that column are the central transducer element of that column; accordingly, the transducer element in the second row of every column is individually connected to a wire for connecting to the channel 01 and receiving an excitation signal from that channel 01. For instance, among the three (rows of) transducer elements in the first column, the transducer element 12 in the second row is the central transducer element, which is individually connected to a wire for connecting to the channel 01 and receiving the excitation signal from that channel 01. Similarly, among the three (rows of) transducer elements in the second column, the transducer element 12 is the central transducer element, which is individually connected to a wire for connecting to the channel 01 and receiving an excitation signal from that channel 01. Again, among the three (rows of) transducer elements in the nth column, the transducer element 12 in the second row is the central transducer element, which is individually connected to a wire for connecting the channel 01 and receiving an excitation signal from that channel 01, where n is a positive integer less than or equal to N. For each column of the transducer elements 12, the transducer elements 12 that are symmetrically positioned around the central element (i.e., the element in the second row of that column) are pairwise connected together and then connected to a wire to receive an excitation signal from the same channel 01. That is, the transducer elements 12 in the first and third rows of each column are pairwise connected together and connected to a wire to receive the excitation signal from the same channel 01. For example, among the three (rows of) transducer elements in the first column, the transducer elements in the first and third rows are symmetrical, and they are connected together and connected to a wire to receive the excitation signal from the same channel 01. Similarly, among the three (rows of) transducer elements in the second column, the transducer elements in the first and third rows are symmetrical, and they are connected together and connected to a wire to receive an excitation signal from the same channel 01. Again, among the three (rows of) transducer elements in the nth column, the transducer elements in the first and third rows are symmetrical, and they are connected together and connected to a wire to receive an excitation signal from the same channel 01, where n is a positive integer less than or equal to N.

In the example shown in FIG. 10, M=5, meaning that the array of transducer elements 11 comprises five rows of transducer elements, with the transducer elements 12 in the third row being the transducer elements in the central row. For each column of the transducer elements 12, the transducer element in the third row of that column is the central transducer element; accordingly, each transducer element in the third row of every column is individually connected by a wire to a channel 01 and receives an excitation signal from that channel 01. For instance, among the five (rows of) transducer elements in the first column, the transducer element 12 in the third row is the central transducer element, which is individually connected by a wire to a channel 01 and receives an excitation signal from that channel 01. Similarly, among the five (rows of) elements in the second column, the transducer element 12 in the third row is the central transducer element, which is individually connected by a wire to a channel 01 and receives an excitation signal from that channel 01. Again, among the five (rows of) transducer elements in the nth column, the transducer element 12 in the third row is the central element, which is individually connected by a wire to a channel 01 and receives an excitation signal from that channel 01, where n is a positive integer less than or equal to N. For each column of transducer elements 12, the transducer elements 12 that are symmetrically positioned with respect to the central transducer element (i.e., the transducer element in the third row) within that column are pairwise connected together and then connected by a wire to receive an excitation signal from the same channel 01. That is, the transducer elements 12 in the first and fifth rows of each column are pairwise connected together and connected by a wire to receive an excitation signal from the same channel 01, and the transducer elements 12 in the second and fourth rows of each column are pairwise connected together and connected by a wire to receive an excitation signal from the same channel 01. For example, among the five (rows of) transducer elements in the first column, the transducer elements in the first and fifth rows are symmetrical, and they are connected together and then connected by a wire to receive an excitation signal from the same channel 01. Similarly, the transducer elements in the second and fourth rows of the first column are symmetrical, and they are connected together and then connected by a wire to receive an excitation signal from the same channel 01. Likewise, among the five (rows of) transducer elements in the second column, the transducer elements in the first and fifth rows are symmetrical, and they are connected together and then connected by a wire to receive an excitation signal from the same channel 01. The transducer elements in the second and fourth rows of the second column are symmetrical, so they are connected together and then connected by a wire to receive an excitation signal from the same channel 01. Again, among the five (rows of) transducer elements in the nth column, the transducer elements in the first and fifth rows are symmetrical, and they are connected together and then connected by a wire to receive an excitation signal from the same channel 01. The transducer elements in the second and fourth rows of the nth column are symmetrical, and they are connected together and then connected by a wire to receive an excitation signal from the same channel 01, where n is a positive integer less than or equal to N.

In some embodiments, M is an even number; among the transducer elements 12 arranged in M rows and N columns, the transducer elements 12 in the (M/2)th row and the (M/2+1)th row are the first-frequency transducer elements 12a, and the transducer elements 12 in the 1st row and the Mth row are the second-frequency transducer elements 12b. In some embodiments, the transducer elements 12 in the 1st to mth rows are the second-frequency transducer elements 12b, the transducer elements in the (M+1−m)th to Mth rows are the second-frequency transducer elements 12b, and the transducer elements 12 in the (m+1)th to (M−m)th rows are the first-frequency transducer elements 12a; wherein m is a positive integer and less than M/.

In some embodiments, M is an even number; and for each column of transducer elements 12: the symmetrical transducer elements 12 within the column are connected in pairs, and a wire is led out from each pair to receive the excitation signal from the same channel 01. When M is an even number, the first row is symmetrical to the last row, the second row is symmetrical to the second-to-last row, and so on. That is, the 1st to the M/2nd rows, in ascending order of row numbers, are symmetrical to the Mth to the (M/2+1)th rows, in descending order of row numbers, respectively.

In some embodiments, the transducer elements 12 in each pair of symmetrical rows are elements of the same frequency.

FIG. 11 illustrates an example where the gray-filled squares represent the first-frequency transducer elements 12a, the white-filled squares represent the second-frequency transducer elements 12b, and the rectangles filled with diagonal lines represent the channel 01. In the example of FIG. 11, M=4, meaning the array of transducer elements 11 comprises four rows of transducer elements, with the first and second rows of transducer elements being symmetrical, as well as the second and third rows of transducer elements. For each column of transducer elements 12, the symmetrical transducer elements 12 within that column are connected in pairs, with each pair leading to a wire for receiving excitation signals from the same channel 01. Specifically, for each column, the transducer elements 12 in the first and fourth rows are connected in pairs, each pair leading to a wire to receive excitation signals from the same channel 01; similarly, the transducer elements 12 in the second and third rows are connected in pairs, each pair leading to a wire to receive excitation signals from the same channel 01. For instance, in the first column of four (rows of) transducer elements, the transducer elements in the first and fourth rows are symmetrical and are connected together, leading to a wire to receive excitation signals from the same channel 01; and the transducer elements in the second and third rows are symmetrical and are connected together, leading to a wire to receive excitation signals from the same channel 01. Similarly, in the second column of four (rows of) transducer elements, the transducer elements in the first and fourth rows are symmetrical and are connected together, leading to a wire to receive excitation signals from the same channel 01; and the transducer elements in the second and third rows are symmetrical and are connected together, leading to a wire to receive excitation signals from the same channel 01. For the nth column of four (rows of) transducer elements, this symmetrical connection and wire leading pattern also applies, where the transducer elements in the first and fourth rows are connected together to receive excitation signals from the same channel 01, and similarly, the transducer elements in the second and third rows are connected together to receive excitation signals from the same Channel 01; where n is a positive integer less than or equal to N.

In some embodiments, the ultrasonic probe 10 is a 1.XD probe, where X is a specific numerical value greater than 0 and less than 2. For example, the 1.XD probe is a 1.25D probe, a 1.5D probe, or a 1.75D probe, that is, the ultrasonic probe 10 is a 1.25D probe, a 1.5D probe, or a 1.75D probe.

In some embodiments, the array of transducer elements 11 of the ultrasonic probe 10 comprises a row of transducer elements 12, wherein the first-frequency transducer elements 12a and the second-frequency transducer elements 12b are arranged at intervals; that is, the first-frequency transducer elements 12a and the second-frequency transducer elements 12b are spaced apart and aligned in a single row. FIG. 12 is an example, where the gray-filled squares represent the first-frequency transducer elements 12a, and the white-filled squares represent the second-frequency transducer elements 12b. In such an example, the ultrasonic probe 10 can be a linear array probe.

The above provides some explanations regarding the arrangement and connection relationships of the array of transducer elements 11 of the ultrasonic probe 10. Next, descriptions about the first-frequency transducer elements 12a and the second-frequency transducer elements 12b will be provided.

In some embodiments, the first frequency or the first frequency range corresponding to the first-frequency transducer elements 12a is greater than the second frequency or the second frequency range corresponding to the second-frequency transducer elements 12b. Specifically, by stating that the first frequency range is greater than the second frequency range, it is meant that the minimum value within the first frequency range exceeds the maximum value within the second frequency range. Similarly, when it is said that the first frequency range is greater than the second frequency, it refers to the minimum value within the first frequency range being greater than the second frequency. Likewise, the statement that the first frequency is greater than the second frequency range implies that the first frequency is higher than the maximum value within the second frequency range.

In some embodiments, the first frequency range corresponding to the first-frequency transducer elements 12a is greater than 7 MHz and less than 15 MHz.

In some embodiments, the first frequency corresponding to the first-frequency transducer elements 12a ranges from 8 MHz to 10 MHz.

In some embodiments, the second frequency range corresponding to the second-frequency transducer elements 12b is greater than 2 MHz and less than 4 MHz.

In some embodiments, the second frequency corresponding to the second-frequency transducer elements 12b ranges from 3 MHz to 4 MHz.

In some embodiments, the first-frequency transducer elements 12a are high-frequency transducer elements; and in some embodiments, the high frequency is greater than 7 MHz and less than 15 MHz.

In some embodiments, the second-frequency transducer elements 12b are low-frequency transducer elements; and in some embodiments, the low frequency is greater than 2 MHz and less than 4 MHz.

The first-frequency transducer elements 12a and the second-frequency transducer elements 12b can be realized using different materials or even different processes.

The above are some descriptions of the ultrasonic probe 10.

The transmit and receive control circuit 20 is configured to control the ultrasonic probe 10 to transmit ultrasonic waves and receive echo signals of the ultrasonic waves. For instance, the transmit and receive control circuit 20 serves to control the ultrasonic probe 10 to transmit ultrasonic waves towards a target region on the one hand, and to control the ultrasonic probe 10 to receive ultrasonic echoes reflected from tissues on the other hand. In some specific embodiments, the transmit and receive control circuit 20 is used to generate transmitting sequences and receiving sequences, which are then output to the ultrasonic probe 10. The transmitting sequences are used to control some or all of the plurality of transducer elements in the ultrasonic probe 10 to transmit ultrasonic waves towards the target region. The parameters of the transmitting sequences include the number of transducer elements used for transmission and transmission parameters for ultrasonic waves (such as pulse amplitude, transmit voltage, transmit frequency, number of transmissions, transmit interval, transmit angle, transmit waveform, transmit aperture, line density, pixel density, and/or focal position, etc.). The receiving sequences are used to control some or all of the plurality of transducer elements to receive ultrasonic echo waves from tissues. The parameters of the receiving sequences include the number of transducer elements used for reception and the reception parameters for the echoes (such as reception angle, depth, etc.). Depending on the different uses of the ultrasonic echoes or the different images generated from the ultrasonic echoes, the parameters for the ultrasonic waves in the transmitting sequences and the parameters for the echoes in the receiving sequences may be different.

The processor 30 is configured to perform signal processing and image processing on the ultrasonic echo signals received by the ultrasonic probe 10 (i.e., echo signals of ultrasound waves). Signal processing refers to one or more steps of processing the echo signals/channel echo data of ultrasonic waves in the signal domain, which may include, for example, analog-to-digital conversion, signal demodulation, amplification, filtering, down-sampling, and/or beamforming. Image processing refers to one or more steps of processing the ultrasonic image data that has undergone signal processing in the image domain, which may include, for example, modulus operation, logarithmic compression, grayscale transformation, and/or other unlisted image processing procedures. In addition, it shall be understood that image processing may also involve image algorithm processing based on the ultrasonic image after it has been generated.

Below is an explanation of the processing steps involved in signal processing and image processing.

The transducer elements of the ultrasonic probe 10 receive echo signals of the ultrasonic waves and convert them into data represented by electrical signals. This data is in the form of analog signals, which are then converted into digital signals through an analog-to-digital conversion process. Signal demodulation may refer to demodulating the input ultrasonic data, where the input ultrasonic data can be a digital signal obtained after the analog-to-digital conversion. Demodulation methods may include: simple demodulation, quadrature demodulation, Hilbert transform demodulation, specific sampling-based demodulation techniques (such as dual sampling, subsampling, or other forms of two or more distinct sampling processes), multiple sampling demodulation, or baseband sampling demodulation, etc. . . . Quadrature demodulation is commonly used, which involves splitting the received echo signal into two paths and multiplying them by cos(ωnTₛ) and sin(ωnTₛ), respectively. The amplification processing step includes: amplifying the ultrasonic data using different amplification factors based on the reception time of the ultrasonic data to compensate for signal attenuation; or, applying different amplification factors based on the location of the ultrasonic data to compensate for signal attenuation. The amplification processing step may follow the signal demodulation process.

The filtering process is typically carried out after signal demodulation to improve signal quality, using, for example, a low-pass filter. Downsampling is employed to reduce the sampling rate of the signal, thereby decreasing computational load. Data normalization can include scaling normalization or standard normalization, which confines the data within a certain range, thus eliminating adverse effects caused by outlier (sample) data.

The process of principal component analysis (PCA) includes: centering the features of the ultrasonic data to obtain processed features, solving for the covariance matrix of these features, calculating the eigenvalues of the covariance matrix, selecting the largest eigenvalues to form eigenvectors, and projecting the ultrasonic data onto these eigenvectors. PCA primarily serves to reduce the dimensionality of the data features.

Data augmentation involves applying translations and/or adding noise to ultrasonic data, which is aimed at improving the accuracy of neural network data processing. For example, when training a neural network with a limited amount of training data, operations such as translating the data and adding noise are performed to augment the dataset, thereby enhancing the accuracy of the neural network.

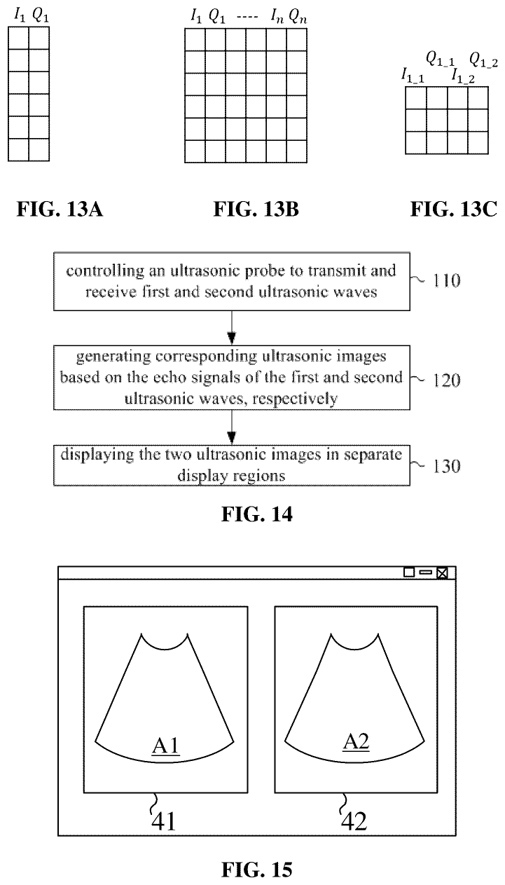

Data rearrangement involves rearranging the ultrasonic data in at least one of the following manners: arranging the demodulated ultrasonic data received by each transducer element of the ultrasonic probe 10 into two columns (one for I data and one for Q data; assuming the data received by a particular transducer element is (Npoint*1), it would be arranged into two columns as shown in FIG. 13A, with I1Q1 representing the two columns of data) or arranging it into one column before demodulation; arranging the ultrasonic echo data (Npoint*2n) received by all active transducer elements after a single ultrasonic emission by the ultrasonic probe 10 into a matrix (as shown in FIG. 13B, an N*2n matrix of I1Q1 . . . InQn, where n is the number of active transducer elements); and dividing the ultrasonic echo data (Npoint*1) received by each transducer element of the ultrasonic probe 10 into a plurality of (e.g., m) parts (e.g., m parts) and then arranging them into a matrix (Npoint/m, 2m, as shown in FIG. 13C with I1-1Q1-1, I1-2Q1-2 data; FIG. 13C illustrates an example where m is 2). It should be noted that in the case of pre-demodulation data, the number of columns in FIGS. 13A to 13C is halved. Additionally, in other examples, the data in FIGS. 13A to 13C can be combined to form three-dimensional or even higher-dimensional data input. The rearranged data is input into a neural network as input data, which can improve the accuracy of the neural network.

The beamforming process refers to the transformation of channel echo data (which can be either radio frequency signals before demodulation or baseband signals after demodulation) from the channel domain (where the data dimensions are: time direction * number of channels * number of transmissions) into beam domain data (i.e., beamformed data, where the data dimensions are: number of longitudinal points * number of transverse points, representing points in actual physical space). Various beamforming procedures can be employed, including but not limited to the Delay and Sum (DAS) procedure, adaptive beamforming procedure, coherence factor beamforming procedure, and so on.

Demodulation, logarithmic compression, and grayscale transformation are processing steps applied to ultrasonic image data in the image domain, which can also be collectively referred to as scan conversion.

Furthermore, specific image processing procedures can be selectively applied to different types of ultrasonic images. For example, for B-mode images, adjustments can be made to parameters such as dynamic range, grayscale mapping, and smoothing; for C-mode images, adjustments can be made to parameters related to smoothing, color mapping, and color priority.

The processor 30 processes the echo signals of ultrasonic waves to ultimately obtain an ultrasonic image for display on the display 40.

In some embodiments, the processor 30 includes, but is not limited to, devices such as those that interpret computer instructions and process data within computer software, such as a central processing unit (CPU), a micro controller unit (MCU), a field-programmable gate array (FPGA), and digital signal processing (DSP) units.

In some embodiments, the processor 30 is configured to perform various computer application programs stored in a computer-readable storage medium, thereby carrying out the corresponding steps and methods.

In some embodiments, the processor 30 can perform the ultrasonic imaging method mentioned herein or steps thereof.

The display 40 can be used to display information, such as parameters and images calculated by the processor 30.

The above are some descriptions of the ultrasonic imaging system 100.

In some embodiments, the ultrasonic imaging system 100 is capable of performing an ultrasonic imaging method, for example, by its processor 30 itself or by controlling other components to execute the ultrasonic imaging method or one or more steps thereof. The ultrasonic imaging method is described below.

Please refer to FIG. 14, the ultrasonic imaging method in some embodiments may include the following steps:

Step 110: controlling an ultrasonic probe 10 to perform transmission and reception of first and second ultrasonic waves. In some examples, step 110 involves controlling the ultrasonic probe 10 to transmit the first ultrasonic waves and the second ultrasonic waves to a first region and a second region of the same tissue imaging plane, respectively, and to receive the echo signals of the first ultrasonic waves and the echo signals of the second ultrasonic waves.

Step 120: generating corresponding ultrasonic images based on the echo signals of the first and second ultrasonic waves, respectively. In some embodiments, step 120 involves generating a first ultrasonic image based on the echo signals of the first ultrasonic waves, and generating a second ultrasonic image based on the echo signals of the second ultrasonic waves.

Step 130: displaying the two ultrasonic images in separate display regions. In some embodiments, step 130 involves displaying a first ultrasonic image in a first display region and a second ultrasonic image in a second display region of the display interface, wherein the first display region and the second display region are two different display regions.

FIG. 15 illustrates an example of the display interface, which comprises a first display region 41 and a second display region 42. The first display region 41 displays a first ultrasonic image A1, while the second display region 42 displays a second ultrasonic image A2.

In some examples, the position and size of the first display region described herein can be adjusted, and similarly, the position and size of the second display region described herein can also be adjusted. In other embodiments, the second display region may be superimposed over the first display region, and the size of the second display region can be larger than, equal to, or smaller than that of the first display region.

By transmitting first ultrasonic waves to a first region and receiving corresponding echo signals, an ultrasonic image of the first region (designated as the first ultrasonic image) can be generated. Concurrently, second ultrasonic waves transmitted to a second region with received echo signals produce an ultrasonic image of the second region (designated as the second ultrasonic image). By designing and arranging the transmission sequence of the first and second ultrasonic waves, it is possible to update the first and second ultrasonic images in real time independently, thus enabling dual real-time ultrasonic imaging of both the first and second regions within the same tissue imaging plane using a single ultrasonic probe 10. Furthermore, the signal/data sources for generating the first and second ultrasonic images are independent.

For example, step 110 involves controlling an ultrasonic probe 10 to alternately transmit first ultrasonic waves and second ultrasonic waves multiple times to the first region and the second region, respectively, and to sequentially receive corresponding echo signals of the first and second ultrasonic waves. Based on the echo signals of the first ultrasonic waves, a first ultrasonic image is generated and updated in real time, while based on the echo signals of the second ultrasonic waves, a second ultrasonic image is generated and updated in real time.

In some instances, the ultrasonic probe 10 transmits one or more rounds of first ultrasonic waves to a first region during one ultrasonic transmission, transmits one or more rounds of second ultrasonic waves to a second region during the next ultrasonic transmission, then transmits one or more rounds of first ultrasonic waves to the first region during the subsequent ultrasonic transmission, and transmits one or more rounds of second ultrasonic waves to the second region during the following ultrasonic transmission, continuously alternating between the transmissions of the first and second ultrasonic waves in this manner. It can be seen that each transmission of the first ultrasonic waves may involve transmitting one or more rounds of first ultrasonic waves to the first region, and each transmission of the second ultrasonic waves may involve transmitting one or more rounds of second ultrasonic waves to the second region. In some embodiments, a frame of a first ultrasonic image can be generated from the echo signals received from each round of transmission of first ultrasonic waves. In some embodiments, a frame of a second ultrasonic image can be generated from the echo signals received from each round of transmission of second ultrasonic waves.

In some embodiments, both the first ultrasonic image and the second ultrasonic image are of the same image mode; for example, they can be one of a two-dimensional grayscale ultrasonic image, a Doppler ultrasonic image, a vector flow image, a super microvascular image, a contrast-enhanced ultrasonic image, a photoacoustic ultrasonic image, or an elasticity image. In other words, both images can be two-dimensional grayscale ultrasonic images, Doppler ultrasonic images, contrast-enhanced ultrasonic images, photoacoustic ultrasonic images, or elasticity images.

In some embodiments, the image display characteristics of the second ultrasonic image are higher than the corresponding image display characteristics of the first ultrasonic image; in some embodiments, the image display characteristics include at least one of image penetration, image contrast, image resolution, and image clarity.

By setting/adjusting the transmission parameters of the first and second ultrasonic waves, the signal processing performed on their respective echo signals in the signal domain, and the image processing performed on their ultrasonic image data in the image domain, the identical image display characteristics of the first and second ultrasonic images can be differentiated. A detailed explanation is provided below.

In some embodiments, step 110 involves controlling an ultrasonic probe 10 to emit first ultrasonic waves based on a first transmission parameter and controlling the same ultrasonic probe 10 to emit second ultrasonic waves based on a second transmission parameter; in some embodiments, step 120 involves performing a first processing on the echo signal of the first ultrasonic waves to generate a first ultrasonic image, wherein the first processing comprises first signal processing and/or first image processing; in some embodiments, step 120 involves performing a second processing on the echo signal of the first ultrasonic waves to generate a second ultrasonic image, wherein the second processing comprises second signal processing and/or second image processing.

It should be noted that the first signal processing and the second signal processing refer to the signal processing mentioned above, which involves one or more steps of processing the echo signals of ultrasonic waves/channel echo data in the signal domain. Examples of such processing may include analog-to-digital conversion, signal demodulation, amplification, filtering, down-sampling and/or beamforming. The first image processing and the second image processing refer to the image processing mentioned above, which involves one or more steps of processing the ultrasonic image data that has undergone signal processing in the image domain. Examples of such processing may include modulus operation, logarithmic compression, grayscale transformation, and/or other unlisted image processing procedures, as well as image algorithm processing targeting ultrasonic images.

In some embodiments, one or more differences among the transmission parameters, signal processing, and image processing result in distinct image display characteristics between the first ultrasonic image and the second ultrasonic image, such that, for example, the image display characteristics of the second ultrasonic image are superior to (such as higher than) the corresponding image display characteristics of the first ultrasonic image. That is, one or more differences among the first transmission parameter and the second transmission parameter, the first signal processing and the second signal processing, and the first image processing and the second image processing lead to differences in the same image display characteristic between the first ultrasonic image and the second ultrasonic image, for example, such that the image display characteristics of the second ultrasonic image are superior to the corresponding image display characteristics of the first ultrasonic image.

In some embodiments, the transmission parameters include at least one of pulse amplitude, transmit voltage, transmit frequency, number of transmissions, transmit interval, transmit angle, transmit waveform, transmit aperture, line density, pixel density, and focal position; that is, the first transmission parameter and the second transmission parameter can each include at least one of pulse amplitude, transmit voltage, transmit frequency, number of transmissions, transmit interval, transmit angle, transmit waveform, transmit aperture, line density, pixel density, and focal position.

In some embodiments, the second transmission parameter is different from the first transmission parameter.

By configuring different transmission parameters, the echo signals of the first ultrasonic waves and the echo signals of the second ultrasonic waves are made different, so that the echo signals of the second ultrasonic waves, compared to the echo signals of the first ultrasonic waves, are more favorable for presenting the same image display characteristic in the ultrasonic image.

Furthermore, by configuring different transmission parameters, the echo signals of the first and second ultrasonic waves are made distinct, significantly enhancing the room for subsequent signal processing and image processing of the two real-time ultrasonic images. This allows for greater differentiation in the same image display characteristics between the two real-time ultrasonic images.

In some embodiments, signal processing may include at least one of analog-to-digital conversion, signal demodulation, amplification, filtering, downsampling, and beamforming; that is, the first signal processing and the second signal processing may each include at least one of analog-to-digital conversion, signal demodulation, amplification, filtering, downsampling, and beamforming.

For example, the first signal processing includes a first beam forming procedure, and the second signal processing includes a second beam forming procedure; accordingly, the difference between the first signal processing and the second signal processing may lie in different first and second beam forming procedures.

For another example, the first signal processing includes a first signal demodulation procedure, and the second signal processing includes a second signal demodulation procedure; accordingly, the difference between the first signal processing and the second signal processing may lie in the different first and second signal demodulation procedures.

In some embodiments, the first signal processing and the second signal processing are different.

By applying different signal processing to the echo signals of the first ultrasonic waves and the echo signals of the second ultrasonic waves, the second ultrasonic image data obtained by processing the echo signals of the second ultrasonic waves with the second signal processing is more favorable for presenting the same image display characteristic in ultrasonic images compared to the first ultrasonic image data obtained by processing the echo signals of the first ultrasonic waves with the first signal processing.

In addition, by applying different signal processing to the echo signals of the first ultrasonic waves and the echo signals of the second ultrasonic waves, the space available for subsequent image processing is significantly enhanced, allowing for greater differentiation in the same image display characteristics between the two real-time ultrasonic images.

In some embodiments, image processing may include procedures such as dynamic range processing, gain adjustment, image enhancement algorithms, and colormap mapping; additionally, the first image processing and the second image processing can employ existing processing methods/algorithms for ultrasonic images in the image domain, as well as future processing methods/algorithms for ultrasonic images in the image domain, without limitation herein.

In some embodiments, the first image processing and the second image processing are different.

It shall be understood that different image processing procedures can be applied either during the stage of generating an ultrasonic image from ultrasonic image data or during the subsequent stage where the already generated ultrasonic image is further processed.

In some embodiments, the frame rate of the second ultrasonic image is greater than that of the first ultrasonic image.

As mentioned above, by setting/adjusting the transmission parameters of the first and second ultrasonic waves, the signal processing performed on their respective echo signals in the signal domain, and the image processing performed on their ultrasonic image data in the image domain, the same image display characteristics of the first and second ultrasonic images can be made different. For example, the image display characteristic of the second ultrasonic image is higher than the corresponding image display characteristic of the first ultrasonic image.

In some examples, the image display characteristic of the second ultrasonic image can be selected and/or set manually by users.

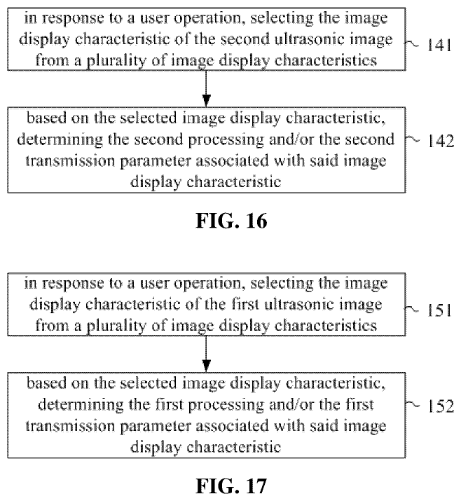

Please refer to FIG. 16. In some embodiments, step 141 involves selecting the image display characteristic of the second ultrasonic image from a plurality of image display characteristics in response to a user operation; and step 142 involves determining the second processing and/or the second transmission parameter associated with the selected image display characteristic. In some embodiments, different image display characteristics are associated with different second processing methods, such as with different second signal processing procedures, with different second image processing procedures, or with both different second signal processing procedures and different second image processing procedures. In some embodiments, different image display characteristics are associated with different second transmission parameters.

Since the image display characteristic of the second ultrasonic image is selected from a plurality of image display characteristics based on a user operation to determine the second processing and/or the second transmission parameter associated with the selected image display characteristic, this approach advantageously optimizes the image display characteristic of the second ultrasonic image, such that it exceeds, for example, a predetermined threshold for the display index, and is superior to the corresponding image display characteristic of the first ultrasonic image.

Please refer to FIG. 17. In some embodiments, step 151 involves selecting the image display characteristic of the first ultrasonic image from a plurality of image display characteristics in response to a user operation; and step 152 involves determining the first processing and/or the first transmission parameter associated with the selected image display characteristic. In some embodiments, different image display characteristics are associated with different first processing methods, such as with different first signal processing procedures, with different first image processing procedures, or with both different first signal processing procedures and different first image processing procedures. In some embodiments, different image display characteristics are associated with different first transmission parameters.

Since the image display characteristic of the first ultrasonic image is selected from a plurality of image display characteristics based on a user operation to determine the first processing and/or the first transmission parameter associated with the selected image display characteristic, this approach advantageously optimizes the image display characteristic of the first ultrasonic image, such that it exceeds, for example, a predetermined threshold for the display index of said image display characteristic, and is superior to the corresponding image display characteristic of the second ultrasonic image.

In some embodiments, the ultrasonic image has a first image display characteristic and a second image display characteristic. In some embodiments, the first ultrasonic image exhibits a first image display characteristic that is superior, for example, exceeding a predefined threshold for the display index of the first image display characteristic, or being better than/superior to the corresponding first image display characteristic of the second ultrasonic image. In some embodiments, the second ultrasonic image exhibits a second image display characteristic that is superior, for example, exceeding a predefined threshold for the display index of the second image display characteristic, or being better than/superior to the corresponding second image display characteristic of the first ultrasonic image. In some embodiments, the first image display characteristic comprises at least one of image penetration, image contrast, image resolution, and image clarity. In some embodiments, the second image display characteristic comprises at least one of image penetration, image contrast, image resolution, and image clarity. In some embodiments, the first image display characteristic and the second image display characteristic are different.

In some embodiments, step 110 involves controlling an ultrasonic probe 10 to transmit first ultrasonic waves according to the first transmission parameter and controlling the same ultrasonic probe 10 to transmit second ultrasonic waves according to the second transmission parameter. In some embodiments, step 120 involves performing a first processing onto the echo signals of the first ultrasonic waves to generate a first ultrasonic image, wherein the first processing comprises first signal processing and/or first image processing. In some embodiments, step 120 involves performing a second processing onto the echo signals of the first ultrasonic waves to generate a second ultrasonic image, wherein the second processing comprises second signal processing and/or second image processing.

In some embodiments, the second transmission parameter is different from the first transmission parameter.

In some embodiments, the first signal processing and the second signal processing are different.

In some embodiments, the first image processing and the second image processing are different.

In some embodiments, one or more differences among one or more of the first and second transmission parameters, the first and second signal processing methods, and the first and second image processing methods result in the same image display characteristics of the first ultrasonic image and the second ultrasonic image being different. For example, such differences allow the first image display characteristic of the first ultrasonic image to be superior, such as exceeding a predetermined threshold for the display index of the first image display characteristic, or being better/higher than the first image display characteristic of the second ultrasonic image. For another example, such differences allow the second image display characteristic of the second ultrasonic image to be superior, such as exceeding a predetermined threshold for the display index of the second image display characteristic, or being better/higher than the second image display characteristic of the first ultrasonic image.

In some embodiments, in response to user operation, an image display characteristic to be optimized is selected from a plurality of image display characteristics as the first image display characteristic of the first ultrasonic image; and based on the selected image display characteristic, the first processing and/or first transmission parameter associated with this image display characteristic are determined. In some embodiments, different image display characteristics are associated with different first processing methods, such as with different first signal processing procedures, with different first image processing procedures, or with both different first signal processing procedures and different first image processing procedures. In some embodiments, different image display characteristics are associated with different first transmission parameters. By selecting an image display characteristic to be optimized from a plurality of image display characteristics as the first image display characteristic of the first ultrasonic image based on user operation, the first processing and/or first transmission parameter associated with this image display characteristic are determined, thereby enabling the first image display characteristic of the first ultrasonic image to be optimized, such as exceeding a predetermined threshold for the display index of the first image display characteristic, or being superior/better than the first image display characteristic of the second ultrasonic image.

In some embodiments, in response to user operation, an image display characteristic to be optimized is selected from a plurality of image display characteristics as the second image display characteristic of the second ultrasonic image; and based on the selected image display characteristic, the second processing and/or second transmission parameter associated with this image display characteristic are determined. In some embodiments, different image display characteristics are associated with different second processing methods, such as with different second signal processing procedures, with different second image processing procedures, or with both different second signal processing procedures and different second image processing procedures. In some embodiments, different image display characteristics are associated with different second transmission parameters. By selecting an image display characteristic to be optimized from a plurality of image display characteristics as the second image display characteristic of the second ultrasonic image based on user operation, the second processing and/or second transmission parameter associated with this image display characteristic are determined, thereby enabling the second image display characteristic of the second ultrasonic image to be optimized, such as exceeding a predetermined threshold for the display index of the second image display characteristic, or being superior/better than the second image display characteristic of the first ultrasonic image.

In some embodiments of the present disclosure, separate image manipulations can be applied to the first ultrasonic image and the second ultrasonic image respectively. These image manipulations include one or more of image rotation, image tilt, image inversion, image magnification, and image parameter measurement.

Further explanation will be provided in conjunction with the first and second regions.

In some embodiments, the first region and the second region are the same region, that is, they are identical.

In some embodiments, the second transmission parameter is different from the first transmission parameter.

In some embodiments, the transmit frequency of the first transmission parameter is lower than the transmit frequency of the second transmission parameter. This can result in higher image resolution for the second ultrasonic image.

In some examples, excitation signals (electrical signals) within the second frequency range are controlled to stimulate the transducer element 12 within the ultrasonic probe 10 to transmit first ultrasonic waves, and excitation signals (electrical signals) within the first frequency range are controlled to stimulate the transducer element 12 within the ultrasonic probe 10 to transmit second ultrasonic waves; wherein the frequency within the second frequency range is lower than that within the first frequency range.

In some examples, excitation signals (electrical signals) within the second frequency range are controlled to stimulate the second-frequency transducer elements 12b within the ultrasonic probe 10 to transmit first ultrasonic waves, and excitation signals (electrical signals) within the first frequency range are controlled to stimulate the first-frequency transducer elements 12a within the ultrasonic probe 10 to transmit second ultrasonic waves; wherein the frequency within the second frequency range is lower than that within the first frequency range.

In some examples, excitation signals (electrical signals) within the second frequency range are controlled to stimulate the first-frequency transducer elements 12a and the second-frequency transducer elements 12b within the ultrasonic probe 10 to transmit first ultrasonic waves, and excitation signals (electrical signals) within the first frequency range are controlled to stimulate the first-frequency transducer elements 12a within the ultrasonic probe 10 to transmit second ultrasonic waves; wherein the frequency within the second frequency range is lower than that within the first frequency range.

In some examples, excitation signals (electrical signals) within the second frequency range are controlled to stimulate the second-frequency transducer elements 12b within the ultrasonic probe 10 to transmit first ultrasonic waves, and excitation signals (electrical signals) within the first frequency range are controlled to stimulate the first-frequency transducer elements 12a and the second-frequency transducer elements 12b within the ultrasonic probe 10 to transmit second ultrasonic waves; wherein the frequency within the second frequency range is lower than that within the first frequency range.

In some embodiments, the transmit frequency of the first transmission parameter is higher than that of the second transmission parameter. This can enhance the image penetration of the second ultrasonic image.

In some examples, excitation signals (electrical signals) within the first frequency range are controlled to stimulate the transducer element 12 within the ultrasonic probe 10 to transmit first ultrasonic waves, and excitation signals (electrical signals) within the second frequency range are controlled to stimulate the transducer elements 12 within the ultrasonic probe 10 to transmit second ultrasonic waves; wherein the frequency within the second frequency range is lower than that within the first frequency range.

In some examples, excitation signals (electrical signals) within the first frequency range are controlled to stimulate the first-frequency transducer elements 12a within the ultrasonic probe 10 to transmit first ultrasonic waves, and excitation signals (electrical signals) within the second frequency range are controlled to stimulate the second-frequency transducer elements 12b within the ultrasonic probe 10 to transmit second ultrasonic waves; wherein the frequency within the second frequency range is lower than that within the first frequency range.

In some examples, excitation signals (electrical signals) within the first frequency range are controlled to stimulate the first-frequency transducer elements 12a and the second-frequency transducer elements 12b within the ultrasonic probe 10 to transmit first ultrasonic waves, and excitation signals (electrical signals) within the second frequency range are controlled to stimulate the second-frequency transducer elements 12b within the ultrasonic probe 10 to transmit second ultrasonic waves; wherein the frequency within the second frequency range is lower than that within the first frequency range.

In some examples, excitation signals (electrical signals) within the first frequency range are controlled to stimulate the first-frequency transducer elements 12a within the ultrasonic probe 10 to transmit first ultrasonic waves, and excitation signals (electrical signals) within the second frequency range are controlled to stimulate the first-frequency transducer elements 12a and the second-frequency transducer elements 12b within the ultrasonic probe 10 to transmit second ultrasonic waves; wherein the frequency within the second frequency range is lower than that within the first frequency range.

In some embodiments, the first signal processing and the second signal processing are different.

In some embodiments, the first image processing and the second image processing are different.

In some embodiments, the second transmission parameter is different from the first transmission parameter, the first signal processing and the second signal processing are the same or different, and the first image processing and the second image processing are the same or different.

In some embodiments, the second transmission parameter is the same as the first transmission parameter, but the first signal processing and the second signal processing are different, while the first image processing and the second image processing are the same or different.

In some embodiments, the first region and the second region are different regions. For example, the second region is a sub-region of the first region. For example, the second region partially overlaps with the first region. For example, the second region and the first region do not overlap at all. Detailed explanations are provided below.

In some embodiments, the second region is a sub-region of the first region.

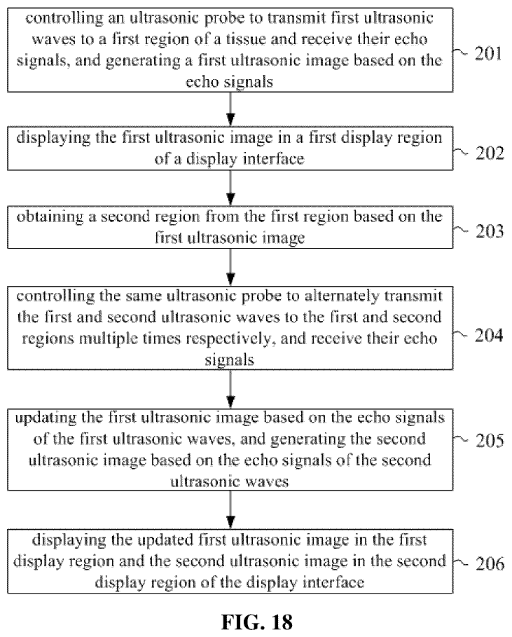

Accordingly, please refer to FIG. 18, some examples of the ultrasonic imaging method may include the following steps:
Step 201: controlling an ultrasonic probe 10 to transmit first ultrasonic waves to a first region of a tissue and receive the echo signals of the first ultrasonic waves, and generating a first ultrasonic image based on the echo signals of the first ultrasonic waves;
Step 202: displaying the first ultrasonic image in a first display region of a display interface;
Step 203: obtaining a second region from the first region based on the first ultrasonic image. Therefore, the second region is a sub-region of the first region, and both the first and second regions are located on the same tissue imaging plane;

In some embodiments, in response to user operation, step 203 involves acquiring a sampling region in the first ultrasonic image displayed in the first display region; and step 203 further involves calculating the second region based on the sampling region.
Step 204: controlling the same ultrasonic probe 10 to alternately transmit the first and second ultrasonic waves respectively to the first and second regions multiple times, and receive the echo signals of the first and second ultrasonic waves;

How to control the ultrasonic probe 10 to alternately emit the first and second ultrasonic waves respectively to the first and second regions multiple times in step 204 can be referred to in the description of step 110 above, which will not be repeated here.
Step 205: updating the first ultrasonic image based on the echo signals of the first ultrasonic waves, and generating the second ultrasonic image based on the echo signals of the second ultrasonic waves; and
Step 206: displaying the updated first ultrasonic image in the first display region of the display interface, and displaying the second ultrasonic image in the second display region of the display interface. In some embodiments, the first display region and the second display region are two different display regions.

It shall be understood that as the ultrasonic probe 10 alternately emits the first and second ultrasonic waves respectively to the first and second regions multiple times, both the first and second ultrasonic images can be updated in real time.

In addition, the first ultrasonic image and the second ultrasonic image are of the same image mode, which can be referred to in the previous description and will not be repeated here.

In some embodiments, the image display characteristic of the second ultrasonic image are higher than the corresponding image display characteristic of the second region in the first ultrasonic image. In some embodiments, the image display characteristic includes at least one of image penetration, image contrast, image resolution, image clarity, and richness of image details.

By setting/adjusting the transmission parameters of the first and second ultrasonic waves, the signal processing performed on their respective echo signals in the signal domain, and the image processing performed on their ultrasonic image data in the image domain, the image display characteristic of the second ultrasonic image can be made higher than the corresponding image display characteristic of the second region in the first ultrasonic image.

In some embodiments, the second transmission parameter is different from the first transmission parameter.

In some embodiments, the first transmission parameter and the second transmission parameter are configured such that the scan density of the second region by the second ultrasonic waves is higher than the scan density of the first region by the first ultrasonic waves; wherein the scan density includes a scanning line density and/or a scanning pixel density.

In some embodiments, the first transmission parameter and the second transmission parameter enable the ultrasonic probe 10 to perform vector scan and linear scan, respectively. For example, vector scan is performed to complete the transmission of the first ultrasonic waves to the first region, and linear scan is performed to complete the transmission of the second ultrasonic waves to the second region. This can be achieved using the 1.XD ultrasonic probe 10 disclosed herein for both vector scan and linear scan.

In some embodiments, the first signal processing and the second signal processing are different.

In some embodiments, the first image processing and the second image processing are different.

In some embodiments, the second transmission parameter is different from the first transmission parameter, the first signal processing and the second signal processing are the same or different, and the first image processing and the second image processing are the same or different.

In some embodiments, the second transmission parameter is the same as the first transmission parameter, but the first signal processing and the second signal processing are different, while the first image processing and the second image processing are the same or different.

In some embodiments, the second region partially overlaps or does not overlap at all with the first region. In some embodiments, the second transmission parameter is different from the first transmission parameter, and the first signal processing and the second signal processing are the same or different, and the first image processing and the second image processing are the same or different. In some embodiments, the second transmission parameter is the same as the first transmission parameter, but the first signal processing and the second signal processing are different, while the first image processing and the second image processing are the same or different.

It shall be noted that in embodiments where the first and second regions are different and the first and second transmission parameters are the same, the transmission parameters do not include focal position.

In some examples, dual real-time ultrasonic images (such as the first ultrasonic image and the second ultrasonic image) can also reuse the same ultrasonic wave and its echo signal, but different signal processing procedures are applied when processing the signals in the signal domain.

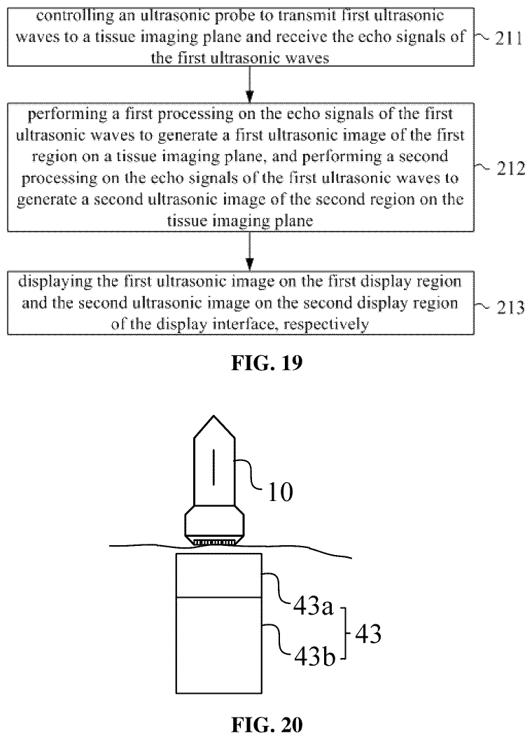

Accordingly, please refer to FIG. 19, some examples of the ultrasonic imaging method may include the following steps:
Step 211: controlling an ultrasonic probe 10 to emit first ultrasonic waves to a tissue imaging plane and receive the echo signals of the first ultrasonic waves;
Step 212: performing a first processing on the echo signals of the first ultrasonic waves to generate a first ultrasonic image of the first region on a tissue imaging plane, and performing a second processing on the echo signals of the first ultrasonic waves to generate a second ultrasonic image of the second region on the tissue imaging plane;

It can be seen that both the first and second regions are located on the same tissue imaging plane.

In some embodiments, the transmit and receive control circuit 20 controls the first-frequency transducer elements 12a and the second-frequency transducer elements 12b to operate jointly as transmitting transducer elements for emitting ultrasonic waves. For instance, the transmit and receive control circuit 20 may generate excitation signals (electrical signals) within a second frequency range to stimulate both the first-frequency transducer elements 12a and the second-frequency transducer elements 12b. For another instance, the transmit and receive control circuit 20 may generate excitation signals (electrical signals) within a first frequency range to stimulate the first-frequency transducer elements 12a, and generate excitation signals (electrical signals) within a second frequency range to stimulate the second-frequency transducer elements 12b.

In some embodiments, please refer to FIG. 20, the first region 43 includes a first sub-region 43a that is proximal to the array of transducer elements 11 and a second sub-region 43b that is distal to the array of transducer elements 11. Herein, “proximal to” and “distal to” the array of transducer elements 11 refer to the positions relative to the array of transducer elements 11 during the scanning process of the ultrasonic probe 10 (i.e., the process of transmitting ultrasonic waves and receiving echo signals).

In some embodiments, the first sub-region 43a refers to a near-field region, and the second sub-region 43b refers to a far-field region.

In some embodiments, the transmit and receive control circuit 20 controls the first-frequency transducer elements 12a as the transmitting transducer elements to emit ultrasonic waves to the first first sub-region 43, and controls the second-frequency transducer elements 13b as the transmitting transducer elements to emit ultrasonic waves to the second sub-region 43.

In some embodiments, the second region is a sub-region of the first region, such as the first sub-region 43a or the second sub-region 43b mentioned above.

In some embodiments, the first processing includes first signal processing and/or first image processing; and the second processing includes second signal processing and/or second image processing.

In some embodiments, the first signal processing and the second signal processing are different.

By applying different signal processing to the echo signals of the first ultrasonic waves, the second ultrasonic image data obtained by processing the echo signals of the first ultrasonic waves with the second signal processing is more favorable for presenting the same image display characteristic in the ultrasonic image compared to the first ultrasonic image data obtained by processing the echo signals of the first ultrasonic waves with the first signal processing.

In addition, by applying different signal processing to the echo signals of the first ultrasonic waves, the space available for subsequent image processing is significantly enhanced, allowing for greater differentiation in image display characteristic between two real-time ultrasonic images.

In some embodiments, signal processing may include at least one of analog-to-digital conversion, signal demodulation, amplification, filtering, downsampling, and beamforming. That is to say, the first signal processing and the second signal processing may include at least one of analog-to-digital conversion, signal demodulation, amplification, filtering, downsampling, and beamforming.

For example, the first signal processing includes a first beam forming procedure, and the second signal processing includes a second beam forming procedure; accordingly, the difference between the first signal processing and the second signal processing may lie in different first and second beam forming procedures.

For another example, the first signal processing includes a first signal demodulation procedure, and the second signal processing includes a second signal demodulation procedure; accordingly, the difference between the first signal processing and the second signal processing may lie in different first and second signal demodulation procedures.

In some embodiments, the first image processing and the second image processing are different.

It shall be understood that different image processing procedures can be applied either during the stage of generating an ultrasonic image from ultrasonic image data or during the subsequent stage where the already generated ultrasonic image is further processed.
Step 213: displaying the first ultrasonic image and the second ultrasonic image in the first display region and second display region, respectively, of the display interface, with the first and second display regions being different. It shall be understood that as the ultrasonic probe 10 continuously emits the first ultrasonic waves to the tissue and receives the echo signals of the first ultrasonic waves, both the first ultrasonic image and the second ultrasonic image can be updated in real time.

In some embodiments, the first ultrasonic image and the second ultrasonic image are of the same image mode. For example, the image mode is one of two- dimensional grayscale image, Doppler image, contrast-enhanced image, photoacoustic image, or elasticity image; in other words, both the first and second ultrasonic images are two-dimensional grayscale images, or both the first and second ultrasonic images are Doppler images, or both the first and second ultrasonic images are contrast-enhanced images, or both the first and second ultrasonic images are photoacoustic images, or both the first and second ultrasonic images are elasticity images.

In some embodiments, the image display characteristic of the second ultrasonic image is higher than the corresponding image display characteristic of the first ultrasonic image. In some embodiments, the image display characteristic include at least one of image penetration, image contrast, image resolution, and image clarity.

In some embodiments, the first region and the second region are the same region, that is, they are identical.

In some embodiments, the first region and the second region are different regions, for example, the second region is a sub-region of the first region. In some embodiments, the second region may be the first sub-region 43a mentioned above. In some embodiments, the second region may be the second sub-region 41b mentioned above.

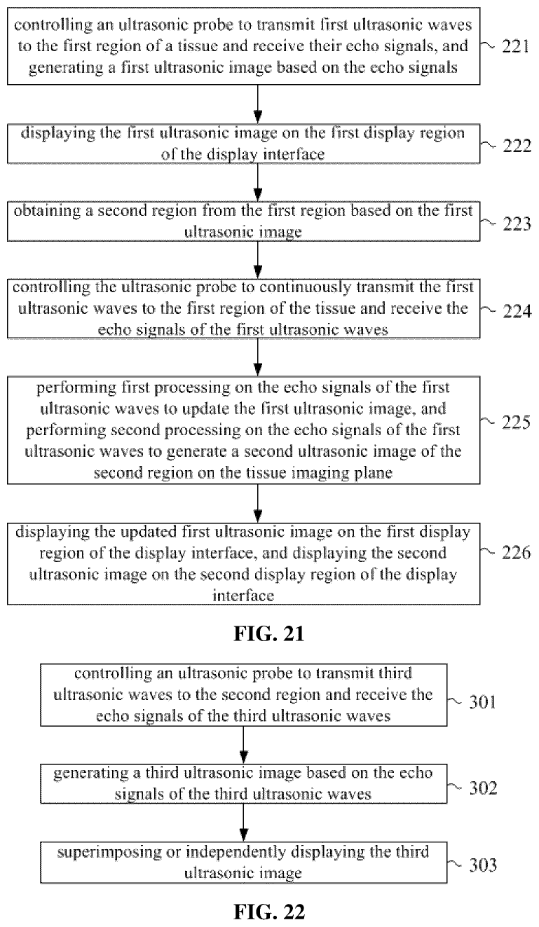

Therefore, please refer to FIG. 21, some examples of the ultrasonic imaging method may include the following steps:
Step 221: controlling an ultrasonic probe 10 to emit first ultrasonic waves to the first region of a tissue and receive the echo signals of the first ultrasonic waves, and generating a first ultrasonic image based on the echo signals of the first ultrasonic waves.

In some examples, the ultrasonic probe 10 performs vector scan. For example, vector scan is performed to complete the transmission of the first ultrasonic waves to the first region. This can be achieved using the 1.XD ultrasonic probe 10 disclosed herein for vector scan.
Step 222: displaying the first ultrasonic image in the first display region of the display interface.
Step 223: obtaining the second region from the first region based on the first ultrasonic image. Accordingly, the second region is a sub-region of the first region, and both the first and second regions are located on the same tissue imaging plane.

In some embodiments, in response to user operation, step 223 involves acquiring a sampling region in the first ultrasonic image displayed in the first display region; and step 223 further involves calculating the second region based on the sampling region.
Step 224: controlling the ultrasonic probe 10 to continuously transmit the first ultrasonic waves to the first region of the tissue and receive the echo signals of the first ultrasonic waves.
Step 225: performing first processing on the echo signals of the first ultrasonic waves to update the first ultrasonic image of the first region, and performing second processing on the echo signals of the first ultrasonic waves to generate a second ultrasonic image of the second region on the tissue imaging plane. It shall be understood that as the ultrasonic probe 10 continuously emits the first ultrasonic waves to the first region of the tissue and receives the echo signals of the first ultrasonic waves, both the first ultrasonic image and the second ultrasonic image can be updated in real time.

In some embodiments, the first processing includes first signal processing and/or first image processing; and the second processing includes second signal processing and/or second image processing. In some embodiments, the first signal processing and the second signal processing are different. In some embodiments, the first image processing and the second image processing are different.
Step 226: displaying the updated first ultrasonic image in the first display region of the display interface, and displaying the second ultrasonic image in the second display region of the display interface, with the first display region and the second display region being two different display regions.

In some embodiments, the first ultrasonic image and the second ultrasonic image are of the same image mode. For example, the image mode is one of two- dimensional grayscale image, Doppler image, contrast-enhanced image, photoacoustic image, or elasticity image; in other words, both the first and second ultrasonic images are two-dimensional grayscale images, or both the first and second ultrasonic images are Doppler images, or both the first and second ultrasonic images are contrast-enhanced images, or both the first and second ultrasonic images are photoacoustic images, or both the first and second ultrasonic images are elasticity images.

In some embodiments, the image display characteristic of the second ultrasonic image is higher than the corresponding image display characteristic of the first ultrasonic image. In some embodiments, the image display characteristic include at least one of image penetration, image contrast, image resolution, and image clarity.

In some embodiments, in response to user operation, the image display characteristic of the second ultrasonic image is selected from a plurality of image display characteristics; and based on the selected image display characteristic, the second processing associated with this image display characteristic is determined. In some embodiments, different image display characteristics are associated with different second processing, such as with different second signal processing, with different second image processing, with both different second signal processing and different second image processing. In some embodiments, image display characteristics are associated with different second transmission parameters.

In some embodiments, in response to user operation, the image display characteristic of the first ultrasonic image is selected from a plurality of image display characteristics; and based on the selected image display characteristic, the first processing associated with this image display characteristic is determined. In some embodiments, different image display characteristics are associated with different first processing, such as with different first signal processing, with different first image processing, with both different first signal processing and different first image processing.

In some embodiments of the present disclosure, separate image manipulations can be applied to the first ultrasonic image and the second ultrasonic image respectively. These image manipulations include one or more of image rotation, image tilt, image inversion, image magnification, and image parameter measurement.

Please refer to FIG. 22, some embodiments of the ultrasonic imaging method may further include the following steps:
Step 301: controlling the same ultrasonic probe 10 to emit third ultrasonic waves to the second region and receive the echo signals of the third ultrasonic waves.
Step 302: generating a third ultrasonic image based on the echo signals of the third ultrasonic waves. In some embodiments, the image mode of the third ultrasonic image is different from that of the second ultrasonic image. For example, the second ultrasonic image is a two-dimensional grayscale image or contrast-enhanced image, and the third ultrasonic image is a Doppler image or elasticity image.
Step 303: displaying the third ultrasonic image. For example, in step 303, the third ultrasonic image is superimposed and displayed on the second ultrasonic image. For example, in step 303, the third ultrasonic image is displayed in the third display region of the display interface, which is a display region different from the first and second display regions.

The present disclosure is illustrated with reference to various exemplary embodiments. However, those skilled in the art may recognize that the exemplary embodiments can be changed and modified without departing from the scope of the present disclosure. For example, various operation steps and components used to execute the operation steps may be implemented in different ways (for example, one or more steps may be deleted, modified, or combined into other steps) according to specific application(s) or any number of cost functions associated with the operation of the system.

In the above embodiments, it may be done in whole or in part by software, hardware, firmware, or any combination thereof. In addition, as understood by those skilled in the art, the principles herein may be reflected in a computer program product on a computer-readable storage medium that is preloaded with computer-readable program code. Any tangible, non-temporary computer-readable storage medium can be used, including magnetic storage devices (hard disks, floppy disks, etc.), optical storage devices (CD-ROMs, DVDs, Blu Ray disks, etc.), flash memory and/or the like. The computer program instructions may be loaded onto a general purpose computer, a special purpose computer, or other programmable data processing device to form a machine, so that these instructions executed on a computer or other programmable data processing device can form a device that realizes a specified function. These computer program instructions may also be stored in a computer-readable memory that can instruct a computer or other programmable data processing device to run in a specific way, so that the instructions stored in the computer-readable memory can form a manufacturing product, including a realization device to achieve a specified function. The computer program instructions may also be loaded onto a computer or other programmable data processing device to execute a series of operating steps on the computer or other programmable device to produce a computer-implemented process, so that instructions executed on the computer or other programmable device can provide steps for implementing a specified function.

Although the principles herein have been shown in various embodiments, many modifications to structures, arrangements, proportions, elements, materials, and components that are specifically adapted to specific environmental and operational requirements may be used without deviating from the principles and scope of the present disclosure. These and other modifications and amendments will be included in the scope of the present disclosure.

The foregoing specific description has been illustrated with reference to various embodiments. However, those skilled in the art will recognize that various modifications and changes can be made without departing from the scope of the present disclosure. Accordingly, the present disclosure is illustrative rather than restrictive, and all such modifications will be included in its scope. Similarly, there are solutions to these and other advantages and problems of the various embodiments as described above. However, the benefits, the advantages, solutions to problems, and any elements that can produce them or make them more explicit should not be interpreted as critical, required, or necessary one. The term “comprise” and any other variations thereof used herein are non-exclusive; accordingly, a process, method, article or device that includes a list of elements may include not only these elements, but also other elements that are not explicitly listed or are not part of said process, method, article or device. In addition, the term “coupling” and any other variations thereof as used herein may refer to physical, electrical, magnetic, optical, communication, functional, and/or any other connection.

Those skilled in the art will realize that many changes can be made to the details of the above embodiments without departing from the basic principles of the present disclosure. The scope of the present disclosure shall therefore be determined in accordance with the following claims.

1. An ultrasonic imaging method, comprising:
controlling an ultrasonic probe to transmit first ultrasonic waves to a first region of a tissue and receive echo signals of the first ultrasonic waves, and generating a first ultrasonic image based on the echo signals of the first ultrasonic waves;
displaying the first ultrasonic image on a first display region of a display interface;
obtaining a second region from the first region based on the first ultrasonic image;
controlling the ultrasonic probe to alternately transmit the first ultrasonic waves and second ultrasonic waves to the first region and the second region for a plurality of times and receive echo signals of the first ultrasonic waves and echo signals of the second ultrasonic waves, updating the first ultrasonic image based on the echo signals of the first ultrasonic waves, and generating a second ultrasonic image based on the echo signals of the second ultrasonic waves; and
displaying the updated first ultrasonic image on the first display region of the display interface, and displaying the second ultrasonic image on a second display region of the display interface;
wherein: the first display region and the second display region are different;
the first ultrasonic image and the second ultrasonic image are of a same image type; and
an image display characteristic of the second ultrasonic image is higher than a corresponding image display characteristic of the second region in the first ultrasonic image, and the image display characteristic comprising at least one of an image penetration, an image contrast, an image resolution, an image clarity, and a richness of image details.

2. The ultrasonic imaging method according to claim 1, wherein
the ultrasonic probe is controlled to transmit the first ultrasonic waves according to a first transmission parameter and the ultrasonic probe is controlled to transmit the second ultrasonic waves according to a second transmission parameter, and the first transmission parameter is the same as the second transmission parameter;
generating and/or updating the first ultrasonic image based on the echo signals of the first ultrasonic waves comprises: performing a first processing on the echo signals of the first ultrasonic waves; and
generating the second ultrasonic image based on the echo signals of the second ultrasonic waves comprises: performing a second processing on the echo signals of the second ultrasonic waves;
wherein: the first processing comprises a first signal processing and/or a first image processing, and the second processing comprises a second signal processing and/or a second image processing; and the first signal processing is different from the second signal processing, and/or, the first image processing is different from the second image processing.

3. The ultrasonic imaging method according to claim 2, wherein
the first signal processing comprises a first beam forming procedure, the second signal processing comprises a second beam forming procedure, and the first beam forming procedure is different from the second beam forming procedure; and/or,
the first signal processing comprises a first signal demodulation procedure, the second signal processing comprises a second signal demodulation procedure, and the first signal demodulation procedure is different from the second signal demodulation procedure.

4. The ultrasonic imaging method according to claim 1, wherein
the ultrasonic probe is controlled to transmit the first ultrasonic waves according to a first transmission parameter, the ultrasonic probe is controlled to transmit the second ultrasonic waves according to a second transmission parameter, and the first transmission parameter is different from the second transmission parameter.

5. The ultrasonic imaging method according to claim 4, wherein the first transmission parameter and the second transmission parameter comprise at least one of: a pulse amplitude, a transmission voltage, a transmission frequency, a number of transmissions, a transmission interval, a transmission angle, a transmission waveform, a transmission aperture, a line density, a pixel density, and a focal position.

6. The ultrasonic imaging method according to claim 4, wherein the first transmission parameter and the second transmission parameter are configured such that a scan density of the second region by the second ultrasonic waves is greater than a scan density of the first region by the first ultrasonic waves, and the scan density comprises a scanning line density and/or a scanning pixel density.

7. The ultrasonic imaging method according to claim 1, wherein obtaining the second region from the first region based on the first ultrasonic image comprises:
in response to a user operation, obtaining a sampling region from the first ultrasonic image displayed on the first display region; and
calculating the second region based on the sampling region.

8. The ultrasonic imaging method according to claim 2, further comprising:
in response to a user operation, selecting the image display characteristic of the second ultrasonic image from a plurality of image display characteristics; and
according to the selected image display characteristic, determining the second processing that is associated with said image display characteristic, wherein different image display characteristics are associated with different second processing; and/or, according to the selected image display characteristic, determining the second transmission parameter that is associated with said image display characteristic, wherein different image display characteristics are associated with different second transmission parameters.

9. An ultrasonic imaging method, comprising:
controlling an ultrasonic probe to transmit first ultrasonic waves and second ultrasonic wave to a first region and a second region of a same tissue imaging plane, respectively, and to receive echo signals of the first ultrasonic waves and echo signals of the second ultrasonic waves;
generating a first ultrasonic image based on the echo signals of the first ultrasonic waves, and generating a second ultrasonic image based on the echo signals of the second ultrasonic waves; and
displaying the first ultrasonic image and the second ultrasonic image on a first display region and a second display region of a display interface, respectively; wherein:
the first display region and the second display region are different,
the first ultrasonic image and the second ultrasonic image are of a same image type, and
an image display characteristic of the second ultrasonic image is higher than a corresponding image display characteristic of the first ultrasonic image, and the image display characteristic comprises at least one of an image penetration, an image contrast, an image resolution, and an image clarity.

10. The ultrasonic imaging method according to claim 9, wherein
the ultrasonic probe is controlled to transmit the first ultrasonic waves according to a first transmission parameter, the ultrasonic probe is controlled to transmit the second ultrasonic waves according to a second transmission parameter, and the first transmission parameter is the same as the second transmission parameter;
generating the first ultrasonic image based on the echo signals of the first ultrasonic waves comprises: performing a first processing on the echo signals of the first ultrasonic waves; and
generating the second ultrasonic image based on the echo signals of the second ultrasonic waves comprises: performing a second processing on the echo signals of the second ultrasonic waves;
wherein: the first processing comprises a first signal processing and/or a first image processing, and the second processing comprises a second signal processing and/or a second image processing; and the first signal processing is different from the second signal processing; and/or, the first image processing is different from the second image processing.

11. The ultrasonic imaging method according to claim 10, wherein
the first signal processing comprises a first beam forming procedure, the second signal processing comprises a second beam forming procedure, and the first beam forming procedure is different from the second beam forming procedure; and/or,
the first signal processing comprises a first signal demodulation procedure, the second signal processing comprises a second signal demodulation procedure, and the first signal demodulation procedure is different from the second signal demodulation procedure.

12. The ultrasonic imaging method according to claim 9, wherein
the ultrasonic probe is controlled to transmit the first ultrasonic waves according to a first transmission parameter, the ultrasonic probe is controlled to transmit the second ultrasonic waves according to a second transmission parameter, and the first transmission parameter is different from the second transmission parameter.

13. The ultrasonic imaging method according to claim 12, wherein the first transmission parameter and the second transmission parameter comprise at least one of: a pulse amplitude, a transmission voltage, a transmission frequency, a number of transmissions, a transmission interval, a transmission angle, a transmission waveform, a transmission aperture, a line density, a pixel density, and a focal position.

14. The ultrasonic imaging method according to claim 9, wherein the second region is a sub-region of the first region.

15. The ultrasonic imaging method according to claim 13, wherein the first transmission parameter and the second transmission parameter are configured such that a scan density of the second region by the second ultrasonic waves is greater than a scan density of the first region by the first ultrasonic waves, and the scan density comprises a scanning line density and/or a scanning pixel density.

16. The ultrasonic imaging method according to claim 13, wherein a frame rate of the second ultrasonic image is greater than a frame rate of the first ultrasonic image.

17. The ultrasonic imaging method according to claim 15, wherein a transmission frequency of the first transmission parameter is lower than a transmission frequency of the second transmission parameter.

18. The ultrasonic imaging method according to claim 9, wherein
in response to a user operation, the image display characteristic of the second ultrasonic image is selected from a plurality of image display characteristics; and
according to the selected image display characteristic, a/the second processing associated with said image display characteristic is determined, with different image display characteristics being associated with different second processing; and/or, according to the selected image display characteristic, a/the second transmission parameter associated with said image display characteristic is determined, with different image display characteristics being associated with different second transmission parameters.

19. The ultrasonic imaging method according to claim 9, wherein the first ultrasonic image and the second ultrasonic image are capable of undergoing independent image manipulations respectively, and said image manipulations comprise one or more of image rotation, image tilt, image inversion, image magnification, and image parameter measurement.

20. The ultrasonic imaging method according to claim 9, further comprising:
controlling the ultrasonic probe to transmit third ultrasonic waves to the second region and receive echo signals of the third ultrasonic waves, generating a third ultrasonic image based on the echo signals of the third ultrasonic waves, the third ultrasonic image and the second ultrasonic image being of different image types; and
superimposing and displaying the third ultrasonic image on the second ultrasonic image, or displaying the third ultrasonic image on a third display region of the display interface, the third display region being different from the first display region and the second display region.
